(19) United States
(12) Patent Application Publication
(10) Pub. No.: US 2025/0212044 A1
(43) Pub. Date: Jun. 26, 2025

(54) DEVICE-TO-DEVICE MITIGATION OF DISRUPTED COMMUNICATION WITH ANALYTE SENSORS

(71) Applicant: Dexcom, Inc., San Diego, CA (US)

(72) Inventors: Brian C. SMITH, San Diego, CA (US); Jason R. CALABRESE, San Diego, CA (US); Peter B. HEDLUND, San Diego, CA (US)

(21) Appl. No.: 18/984,738

(22) Filed: Dec. 17, 2024

Related U.S. Application Data
(60) Provisional application No. 63/612,906, filed on Dec. 20, 2023.

(51) Int. Cl. H04W 28/02; A61B 5/00; H04W 84/18

(52) U.S. Cl. CPC H04W 28/0231; A61B 5/0004; H04W 84/18

(57) ABSTRACT

In some embodiments, one general aspect includes a method of mitigating disrupted communication with an analyte sensor. The method includes receiving, at a first device, analyte measurements from the analyte sensor. The method also includes receiving, at the first device, an indication of a communication disruption between the analyte sensor and a second device. The method also includes, responsive to the indication, at the first device, facilitating transfer of at least a portion of the analyte measurements from the first device to the second device.

### CROSS-REFERENCE TO RELATED APPLICATIONS

This application claims priority to and benefit of U.S. Provisional Patent Application No. 63/612,906, filed Dec. 20, 2023, which is hereby expressly incorporated by reference herein in its entirety as if fully set forth below and for all applicable purposes.

### INTRODUCTION

Diabetes is a metabolic condition affecting hundreds of millions of people. For these people, monitoring blood glucose levels and regulating those levels to be within an acceptable range is important not only to mitigate long-term issues such as heart disease and vision loss, but also to avoid the effects of hyperglycemia and hypoglycemia. Maintaining blood glucose levels within an acceptable range can be challenging, as glucose levels are almost constantly changing over time and in response to everyday events, such as eating or exercising. Advances in medical technologies have enabled development of various systems for monitoring analytes such as blood glucose, including continuous analyte monitoring (CAM) systems such as continuous glucose monitoring (CGM) systems, which measure and record glucose concentrations in substantially real-time. CAM systems are important tools for users of these systems to ensure that measured analyte values are within the acceptable range.

### SUMMARY

In some embodiments, one general aspect includes a method of mitigating disrupted communication with an analyte sensor. The method includes receiving, at a first device, analyte measurements from the analyte sensor. The method also includes receiving, at the first device, an indication of a communication disruption between the analyte sensor and a second device. The method also includes, responsive to the indication, at the first device, facilitating transfer of at least a portion of the analyte measurements from the first device to the second device.

In some embodiments, another general aspect includes a method of mitigating disrupted communication with an analyte sensor. The method includes receiving, at a first device, analyte measurements from the analyte sensor. The method also includes determining a communication status of a second device. The method also includes, responsive to the communication status indicating a communication disruption between the second device and the analyte sensor, at the first device, facilitating transfer of at least a portion of the analyte measurements from the first device to the second device.

In some embodiments, another general aspect includes a system for mitigating disrupted communications. The system includes an analyte sensor configured to generate measurements associated with an analyte level of a patient. The system also includes a device in communication with the analyte sensor. The device is configured to receive analyte measurements from the analyte sensor. The device is further configured to receive an indication of a communication disruption between the analyte sensor and another device. The device is further configured, responsive to the indication, to facilitate transfer of at least a portion of the analyte measurements from the device to the other device.

In some embodiments, another general aspect includes a computer-program product. The computer-program product includes a non-transitory computer-usable medium having computer-readable program code embodied therein. The computer-readable program code is adapted to be executed to implement a method of mitigating disrupted communication with an analyte sensor. The method includes receiving, at a first device, analyte measurements from the analyte sensor. The method also includes determining a communication status of a second device. The method also includes, responsive to the communication status indicating a communication disruption between the second device and the analyte sensor, at the first device, facilitating transfer of at least a portion of the analyte measurements from the first device to the second device.

### BRIEF DESCRIPTION OF THE DRAWINGS

FIG. 1A illustrates an example of a therapy management system, in accordance with certain embodiments.

FIG. 1B illustrates an example analyte sensor system including an example continuous analyte sensor(s) with sensor electronics, in accordance with certain embodiments.

FIG. 2 illustrates example inputs and example outputs that are generated based on the inputs, in accordance with certain embodiments.

FIG. 3 illustrates an example of a system for sharing a patient's health data between devices and/or with one or more other users, in accordance with certain embodiments.

FIG. 4A illustrates an example of a process for device-to-device mitigation of disrupted communication with analyte sensors, in accordance with certain embodiments.

FIG. 4B illustrates an example of a process for device-to-device mitigation of disrupted communication with analyte sensors, in accordance with certain embodiments.

FIG. 5A illustrates an example of a process for executing a device-to-device transmission flow, in accordance with certain embodiments.

FIG. 5B is a sequence diagram illustrating example interactions among an analyte sensor system and display devices, in accordance with certain embodiments.

FIG. 5C illustrates an example of a process for optimizing communication between devices during execution of a device-to-device transmission flow, in accordance with certain embodiments.

FIG. 6A illustrates an example of a process for executing a device-to-device transmission flow, in accordance with certain embodiments.

FIG. 6B is a sequence diagram illustrating example interactions between an analyte sensor system and display devices, in accordance with certain embodiments.

FIG. 7A illustrates an example of a process for executing a device-to-device transmission flow via a mesh network, in accordance with certain embodiments.

FIG. 7B is a sequence diagram illustrating example interactions among an analyte sensor system, display devices, and a mesh device, in accordance with certain embodiments.

FIG. 8 illustrates an example of a process for anticipatory mitigation of a predicted communication disruption, in accordance with certain embodiments.

FIG. 9 illustrates an example of a process for de-duplicating data, in accordance with certain embodiments.

FIG. 10 is a block diagram depicting a computer system configured for optimizing transmission of analyte data in multi-receiver environments, in accordance with certain embodiments.

### DETAILED DESCRIPTION

In a continuous analyte monitoring system, an analyte sensor measures analyte levels of a patient. The analyte sensor can be, or can include, a continuous analyte monitor (CAM) that continuously monitors analytes in a bodily fluid of the patient. The analyte sensor can communicate the raw sensor measurements to a transmitter, which can then transmit corresponding analyte values to a host receiver device (e.g., a smartphone) for presentation, alerting, and/or storage. The patient can thereby use their device to monitor their health substantially in real-time.

A connectivity issue between a host receiver device and the analyte transmitter, periodically referred to herein as a communication “disruption” or “interruption,” may prevent the host receiver device from receiving analyte data for a period of time. In some cases, the disruption may result from an excessive distance between the analyte sensor and the host receiver device. For example, the host may leave their smartphone out of range of their sensor during certain activities such as running or swimming. In other cases, the disruption may be due to receiver malfunction, power issues (e.g., battery depletion), etc.

In certain aspects, when the host receiver device re-establishes communications with the analyte sensor after the connectivity issue is resolved, backfilling can occur, where data cached at the analyte sensor during the period of disruption is sent to the receiving device upon the reconnection of the two devices. However, the analyte sensor often has limited storage and computing resources, for example, due to its relatively small size and battery capacity. Therefore, backfilling can be a significant resource burden for the analyte sensor. At times, backfilling may be technically infeasible, for example, if a length of the disruption exceeds the sensor's ability to cache data.

Additionally, such communication disruptions can compromise patient health. The display of real-time data is typically of paramount importance to a patient and other users, such as family members or caregivers, as it informs them of the patient's current analyte condition and lets them determine whether one or more actions need to be taken to adjust current analyte levels. If, for example, a user's display device is unable to immediately alert the user when the patient is experiencing a hypoglycemic or hyperglycemic episode, corrective action may be delayed indefinitely, which may increase the severity and length of the episode and may negatively impact health of the patient.

Moreover, such communication disruptions can create gaps in monitoring and reporting. For example, if the analyte sensor is not configured to backfill data, or if the length of the disruption exceeds the analyte sensor's ability to cache and backfill data, the user's display device may not be able to fully report on analyte data from the period of disruption. The device may be unable to identify and report, for example, that the patient's analyte levels were dangerously impacted by an activity (e.g., trending towards hypoglycemia during basketball practice, a swim, a run, etc.). This gap in reporting may result in the patient or other user not being aware of the timing of a severe risk, which may substantially increase the probability of a recurrence during future activities.

In certain aspects, multiple receiver devices (e.g., display devices such as a smartphone, smartwatch, tablet, augmented reality (AR) and/or virtual reality (VR) headset, etc.) can each be configured to directly communicate with, and obtain analyte data from, a single analyte sensor. For example, the analyte sensor may be in direct communication with both a first device (such as a smartwatch) and a second device (such as a smartphone). The analyte sensor can send analyte data such as analyte measurements directly to the first device and directly to the second device. The first device and/or the second device may both capture and then forward this data via one or more communication methods (e.g., cellular, Wi-Fi, Bluetooth or Bluetooth Low Energy (BLE), near-field communication (NFC) and/or the like) to another local network, device, network application, or application in a cloud.

In a scenario where communications between the second device and the analyte sensor are interrupted, but communications between the first device and the sensor are maintained, the first device may cache or buffer all or a portion of the analyte data produced at the sensor. In some aspects, the sensor and the first device may each cache some portion of the aforementioned analyte data, which portions may differ, for example, in type and/or granularity. For example, the first device may store data at more frequent time intervals than the sensor, may store data at a greater level of detail than the sensor, and/or may store additional or different metadata than the sensor (e.g., location data or other obtained metadata). Alternatively, in some aspects, the first device may cache some or all of the analyte data and the analyte sensor may not cache any data. Alternatively, in some aspects, the first device may cache some or all of the analyte data and the analyte sensor may cache less data than the first device and/or may cache less data than the analyte sensor normally caches (e.g., less metadata than is normally cached). In addition, or alternatively, caching responsibility may be split between the first device and the analyte sensor, for example, such that the first device caches a first portion of the analyte data and the analyte sensor caches a second portion of the analyte data that is different from the first portion.

In certain aspects, various technical solutions described herein create a “triangle of success” between a first device (e.g., smartwatch), a second device (e.g., a smartphone), and an analyte sensor. The first and second devices can receive data (e.g., analyte measurements) from the sensor and/or the other device within the triangle. This increases each device's chances of capturing data, minimizing lost data and the subsequent need to perform gap-filling (thereby improving the performance of each device). The transfer of data between the first device and second device may be periodically referred to herein as “side-filling,” in contrast to “back-filling” performed between a sensor and a device. Although certain illustrative examples are described herein in terms of two devices and an analyte sensor forming a “triangle of success,” it should be appreciated that similar reliability and redundancy can be achieved with four, five, six, or any other suitable number devices that are each operable to communicate with an analyte sensor and each other.

In some aspects, sharing data between devices can involve tracking a state of each device and maintaining health data identifiers, such as time stamps, to prevent duplicate data. These identifiers may be maintained by one or more of the devices and may be used to de-duplicate data by recognizing and removing duplicate identifiers associated with records within the health data.

In some aspects, instead of or in addition to a smartwatch as an alternate device, additional devices may be in direct or indirect communication with the sensor. These devices can include, for example, an insulin pump, a thermostat in a house that is connected to a communications device such as a router in the house, which router then forwards the data to other devices. In this way, a mesh network can be created that enables health data to be shared across devices to reach a desired device (e.g., a phone with an analyte monitoring application).

Advantageously, in certain aspects, various solutions described herein can establish a protocol that prioritizes or maximizes the possibility of displaying real-time analyte data to the user when connectivity issues are encountered. In certain aspects, various solutions described herein can expand data “filling” functionality to allow additional devices to assist with data caching and recovery in response, for example, to a communication disruption.

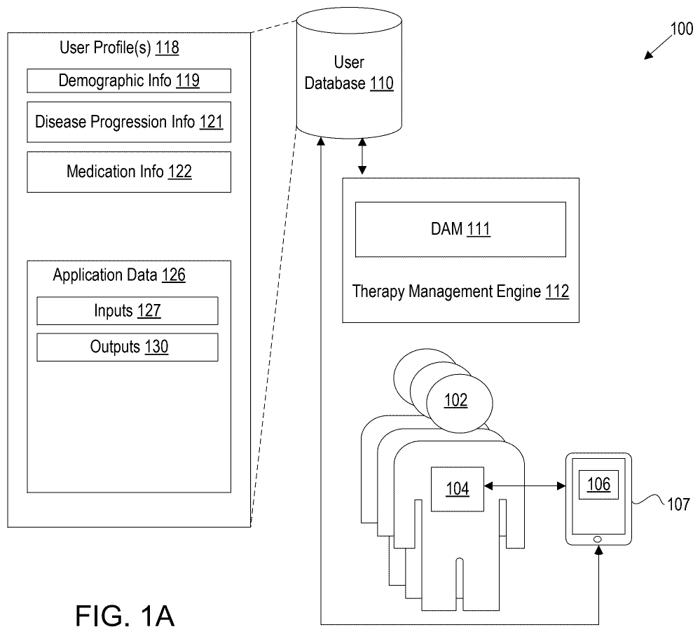

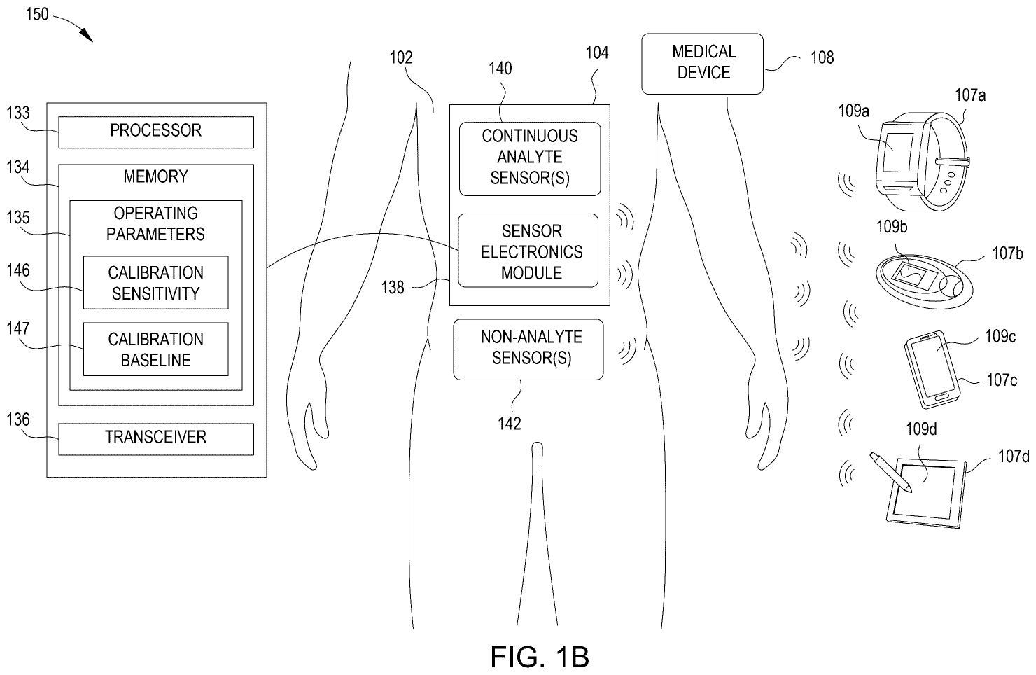

The techniques described herein for device-to-device mitigation of disrupted communication with analyte sensors are described more fully herein with respect to FIGS. 1A-B, 2-4, 5A-C, 6A-B, 7A-B, and 8-9 below. Note that although certain aspects herein are described with respect to the management of diabetes, a continuous glucose monitoring (CGM) system, a CGM application, and the transmission of analyte measurements between devices, the protocols and techniques described herein are similarly applicable to any type of health management system that includes any type of analyte sensor (e.g., lactate sensor, ketone sensor, etc.), any type of health management application, and/or transmission of any type of analyte data. For example, a lactate sensor may be used to monitor for events indicative of sepsis based on lactate thresholds, or a ketone sensor may be used to monitor for ketoacidosis based on ketone thresholds. Additionally, note that, hereinafter, although certain aspects described herein may periodically refer to an analyte monitoring device performing various techniques described herein for transmitting analyte data, it is the transmitter in the analyte monitoring device that performs the various techniques described herein for transmitting analyte data.

As used herein, the term “continuous” analyte monitoring refers to monitoring one or more analytes in a fully continuous, semi-continuous, periodic manner, which results in a data stream of analyte values over time. A data stream of analyte values over time is what allows for meaningful data and insights to be derived using the algorithms described herein for device-to-device mitigation of disrupted communication with analyte sensors. In other words, single point-in-time measurements collected as a result of a patient visiting their health care professional every few months results in sporadic data points (e.g., that are, at best, months apart in timing) that cannot form the basis of any meaningful data or insight to be derived. As such, without the continuous analyte monitoring system of the embodiments herein, it is simply impossible to continuously perform device-to-device mitigation of disrupted communication with analyte sensors, as described herein.

Further, the data stream of analyte values collected over time, with the continuous analyte monitoring system presented herein, include real-time analyte values, which allows for deriving meaningful data and insight in real-time using the systems and algorithms described herein. The derived real-time data and insight in turn allows for device-to-device mitigation of disrupted communication with analyte sensors. Real-time analyte values herein refer to analyte values that become available and actionable within seconds or minutes of being produced as a result of at least one sensor electronics module of the continuous analyte monitoring system (1) converting sensor current(s) (i.e., analog electrical signals) generated by the continuous analyte sensor(s) into sensor count values, (2) calibrating the count values to generate at least glucose and/or other analyte concentration values using calibration techniques described herein to account for the sensitivity of the continuous analyte sensor(s), and (3) transmitting measured glucose and/or other analyte concentration data, including glucose and/or other analyte concentration values, to a display device via wireless connection.

For example, the at least one sensor electronics module may be configured to sample the analog electrical signals at a particular sampling period (or rate), such as every 1 second (1 Hz), 5 seconds, 10 seconds, 30 seconds, 1 minute, 3 minutes, 5 minutes, etc., and to transmit the measured glucose and/or other analyte concentration data to a display device at a particular transmission period (or rate), which may be the same as (or longer than) the sampling period, such as every 1 minute (0.016 Hz), 5 minutes, 10 minutes, etc.

The real-time analyte data that is continuously generated by the continuous analyte monitoring system described herein, therefore, allows the therapy management system herein to perform device-to-device mitigation of disrupted communication with analyte sensors, in real-time, which is technically impossible to perform using existing or conventional techniques or systems. Further, because of the real-time nature of this data, it is also humanly impossible to continuously process a real-time data stream of analyte values over time to derive meaningful data and insight using the algorithms and systems described herein to perform device-to-device mitigation of disrupted communication with analyte sensors. In other words, deriving meaningful data and insight from a stream of real-time data that is continuously generated, processed, calibrated, and analyzed, using the algorithms and systems described herein, is not a task that can be mentally performed. For example, executing the algorithms described in relation to FIGS. 1A-B, 2-4, 5A-C, 6A-B, 7A-B and 8-9, in real-time and on a continuous basis, which would involve using a stream of real-time data that is continuously generated by a patient's continuous analyte monitoring system and/or significantly large amount of population data (e.g., hundreds or thousands of data points for each one of thousands or millions of patients in the patient population) is not a task that can be mentally performed, especially in real-time at times.

Further, certain embodiments herein are directed to a technical solution to a technical problem associated with analyte sensor systems. In particular, each analyte sensor system that is manufactured by a sensor manufacturer might perform slightly differently. As such, there might be inconsistencies between sensors and the measurements they generate once in use. Accordingly, certain embodiments herein are directed to determining the performance of an analyte sensor system during a manufacturing calibration process (in vitro), which includes quantifying certain sensor operating parameters, such as a calibration slope (also known as calibration sensitivity), a calibration baseline, etc.

Generally, calibration sensitivity refers to the amount of electrical current produced by an analyte sensor of an analyte sensor system when immersed in a predetermined amount of a measured analyte. The amount of electrical current may be expressed in units of picoAmps (pA) or counts. The amount of measured analyte may be expressed as a concentration level in units of milligrams per deciliter (mg/dL), and the calibration sensitivity may be expressed in units of pA/(mg/dL) or counts/(mg/dL). The calibration baseline refers to the amount of electrical current produced by the analyte sensor when no analyte is detected, and may be expressed in units of pA or counts.

The calibration sensitivity, calibration baseline, and other information related to the sensitivity profile for the analyte sensor system may be programmed into the sensor electronics module of the analyte sensor system during the manufacturing process, and then used to convert the analyte sensor electrical signals into measured analyte concentration levels. For example, the calibration slope (calibration sensitivity) may be used to predict an initial in vivo sensitivity (M₀) and a final in vivo sensitivity (M_{f}), which are programmed into the sensor electronics module and used to convert the analyte sensor electrical signals into measured analyte concentration levels.

In certain embodiments, during in vivo use, the sensor electronics module of an analyte sensor system samples the analog electrical signals produced by the analyte sensor to generate analyte sensor count values, and then determines the measured analyte concentration levels based on the analyte sensor count values, the initial in vivo sensitivity (M₀), and the final in vivo sensitivity (M_{f}). For example, measured analyte concentration levels may be determined using a sensitivity function M(t) that is based on the initial in vivo sensitivity (M₀) and the final in vivo sensitivity (M_{f}). The sensitivity function M(t) may expressed in several different ways, such as a simple correction factor that is not dependent on elapsed time (tᵢ) of in vivo use, a linear relationship between sensitivity and time (tᵢ), an exponential relationship between sensitivity and time (tᵢ), etc. Equation 1 presents one technique for determining a measured analyte concentration level (ACL) from an analyte sensor count value (count) at a time tᵢ:

$$ACL = count / M ( {t}_{i} ) \qquad Eq . 1$$

A calibration baseline (baseline) may also be used to determine a measured analyte concentration level (ACL) from an analyte sensor count value (count) at a time tᵢ, and Equation 2 presents one technique:

$$ACL = ( count - baseline ) / M ( {t}_{i} ) \qquad Eq . 2$$

FIG. 1A illustrates an example of a therapy management system 100 for device-to-device mitigation of disrupted communication with analyte sensors, in accordance with certain embodiments of the disclosure. The therapy management system 100 may be utilized for generating and presenting information related to user health, for example, using various user interfaces associated with system 100. Each user of system 100, such as user 102, may interact with a mobile health application, such as mobile health application (“application”) 106 (e.g., a diabetes intervention application that provides therapy management guidance), and/or a health monitoring device, such as an analyte sensor system 104 (e.g., a glucose monitoring system). User 102, in certain embodiments, may be the patient or, in some cases, the patient's caregiver. In the embodiments described herein, the user is assumed to be the patient for simplicity only, but is not so limited. As shown, system 100 may include an analyte sensor system 104, a display device 107 that executes application 106, a therapy management engine 112, and a user database 110.

Analyte sensor system 104 may be configured to generate time-series data, such as analyte measurements (e.g., sensor data), for the user 102, e.g., on a continuous basis, and transmit the analyte measurements to the display device 107 for use by application 106. In some embodiments, the analyte sensor system 104 may transmit the analyte measurements to the display device 107 through a wireless connection (e.g., Bluetooth connection). In certain embodiments, display device 107 is a smart phone. However, in certain embodiments, display device 107 may instead be any other type of computing device such as a laptop computer, a smartwatch, a tablet, or any other computing device capable of executing application 106.

Note that, while in certain examples the analyte sensor system 104 is assumed to be a glucose monitoring system, analyte sensor system 104 may operate to monitor one or more additional or alternative analytes. As discussed, the term “analyte” as used herein is a broad term, and is to be given its ordinary and customary meaning to a person of ordinary skill in the art (and is not to be limited to a special or customized meaning), and refers without limitation to a substance or chemical constituent in the body or a biological sample (e.g., bodily fluids, including, blood, serum, plasma, interstitial fluid, cerebral spinal fluid, lymph fluid, ocular fluid, saliva, oral fluid, urine, excretions, or exudates).

Analytes can include naturally occurring substances, artificial substances, metabolites, and/or reaction products. In some embodiments, the analyte measured and used by the devices and methods described herein may include albumin, alkaline phosphatase, alanine transaminase, aspartate aminotransferase, bilirubin, blood urea nitrogen, calcium, CO2, chloride, creatinine, glucose, gamma-glutamyl transpeptidase, hematocrit, lactate, lactate dehydrogenase, magnesium, oxygen, pH, phosphorus, potassium, ketones, sodium, total protein, uric acid, metabolic markers, and/or drugs.

Other analytes are contemplated as well, including but not limited to acetaminophen, dopamine, ephedrine, terbutaline, ascorbate, uric acid, oxygen, d-amino acid oxidase, plasma amine oxidase, xanthine oxidase, NADPH oxidase, alcohol oxidase, alcohol dehydrogenase, pyruvate dehydrogenase, diols, Ros, NO, bilirubin, cholesterol, triglycerides, gentisic acid, ibuprophen, L-Dopa, methyl dopa, salicylates, tetracycline, tolazamide, tolbutamide, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments.

The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA).

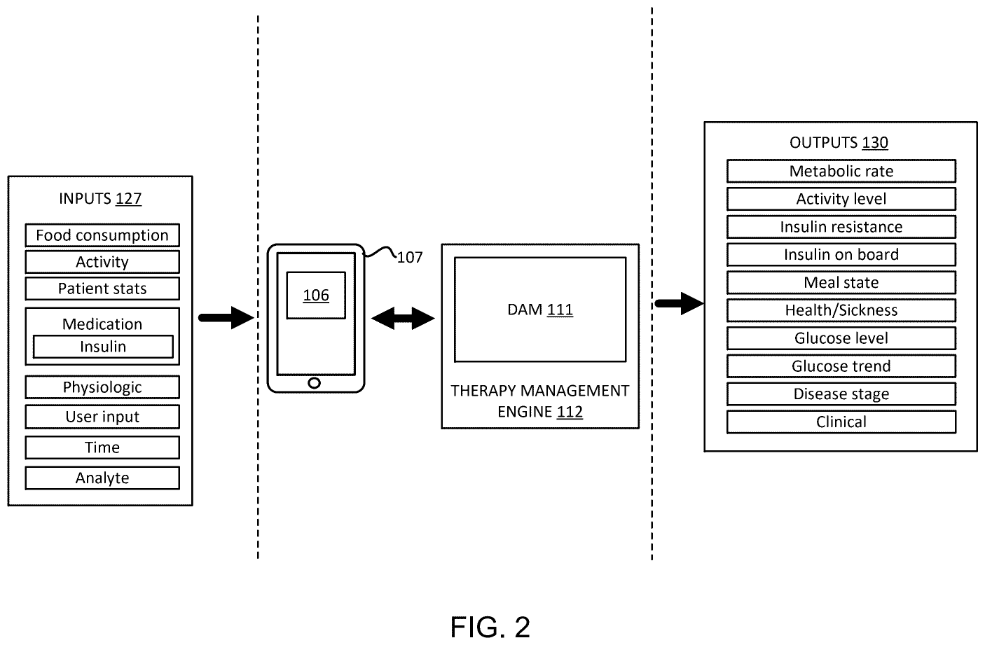

Application 106 may be a mobile health application that is configured to receive and analyze time-series data, including analyte measurements, from the analyte sensor system 104 and/or other devices, as described in greater detail relative to FIGS. 1B and 2. In some embodiments, application 106 may transmit analyte measurements received from the analyte sensor system 104 to a user database 110 (and/or the therapy management engine 112), and the user database 110 (and/or the therapy management engine 112) may store the analyte measurements in a user profile 118 of user 102 for processing and analysis, for example, by the therapy management engine 112. In some embodiments, application 106 may store the analyte measurements in a user profile 118 of user 102 locally for processing and analysis, for example, by the therapy management engine 112.

In certain embodiments, therapy management engine 112 refers to a set of software instructions with one or more software modules, including a data analysis module (DAM) 111. In some embodiments, therapy management engine 112 executes entirely on one or more computing devices in a private or a public cloud. In some other embodiments, therapy management engine 112 executes partially on one or more local devices, such as display device 107 (e.g., via application 106) and/or analyte sensor system 104, and partially on one or more computing devices in a private or a public cloud. In some other embodiments, therapy management engine 112 executes entirely on one or more local devices, such as display device 107 (e.g., via application 106) and/or analyte sensor system 104.

In certain embodiments, DAM 111 of therapy management engine 112 may be configured to receive and/or process a set of inputs 127 (described in more detail below) (also referred to herein as “input data”) to determine one or more outputs 130 (also referred to herein as “metrics data”). Inputs 127 may be stored in the user profile 118 in the user database 110. DAM 111 can fetch inputs 127 from the user database 110 and compute a plurality of outputs 130 which can then be stored as application data 126 in the user profile 118. Such outputs 130 may include health-related metrics.

In certain embodiments, application 106 is configured to take as input information relating to user 102 and store the information in a user profile 118 for user 102 in user database 110. For example, application 106 may obtain and record user 102's demographic info 119, disease progression info 121, and/or medication info 122 in user profile 118. In certain embodiments, demographic info 119 may include one or more of the user's age, body mass index (BMI), ethnicity, gender, etc. In certain embodiments, disease progression info 121 may include information about the user 102's disease, such as, for diabetes, whether the user is Type I, Type II, pre-diabetes, or whether the user has gestational diabetes. In certain embodiments, disease progression info 121 also includes the length of time since diagnosis, the level of disease control, level of compliance with disease management therapy, predicted pancreatic function, other types of diagnosis (e.g., heart disease, obesity) or measures of health (e.g., heart rate, exercise, stress, sleep, etc.), and/or the like. In certain embodiments, medication info 122 may include information about the amount and type of medication taken by user 102, such as insulin or non-insulin diabetes medications and/or non-diabetes medication taken by user 102.

In certain embodiments, application 106 may obtain demographic info 119, disease progression info 121, and/or medication info 122 from the user 102 in the form of user input or from other sources. In certain embodiments, as some of this information changes, application 106 may receive updates from the user 102 or from other sources. In certain embodiments, user profile 118 associated with the user 102, as well as other user profiles associated with other users are stored in a user database 110, which is accessible to application 106, as well as to the therapy management engine 112, over one or more networks (not shown).

In certain embodiments, application 106 collects inputs 127 through user 102 input and/or a plurality of other sources, including analyte sensor system 104, other applications running on display device 107, and/or one or more other sensors and devices. In certain embodiments, such sensors and devices include one or more of, but are not limited to, an insulin pump, other types of analyte sensors, sensors or devices provided by display device 107 (e.g., accelerometer, camera, global positioning system (GPS), heart rate monitor, etc.) or other user accessories (e.g., a smartwatch), or any other sensors or devices that provide relevant information about the user 102. In certain embodiments, user profile 118 also stores application configuration information indicating the current configuration of application 106, including its features and settings.

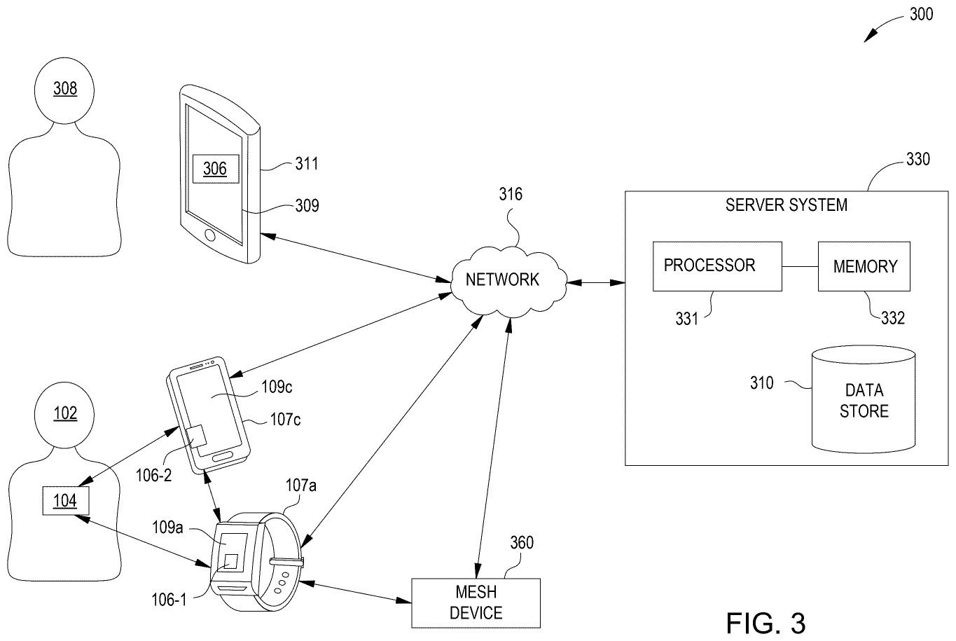

User database 110, in some embodiments, refers to a storage server that may operate in a public or private cloud. User database 110 may be implemented as any type of data store, such as relational databases, non-relational databases, key-value data stores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed. In some aspects, user database 110 may be synchronized with local databases on one or more devices, such as a local database on the display device 107 and/or local databases on one or more mesh devices. Examples of mesh devices will be described relative to FIG. 3.

User database 110 may include other user profiles 118 associated with a plurality of other users served by therapy management system 100. More particularly, similar to the operations performed with respect to the user 102, the operations performed with respect to these other users may utilize an analyte monitoring system, such as analyte sensor system 104, and also interact with the same application 106, copies of which execute on the respective display devices of the other users 102. For such users, user profiles 118 are similarly created and stored in user database 110.

FIG. 1B is a diagram 150 conceptually illustrating an example continuous analyte sensor system 104 including example continuous analyte sensor(s) with sensor electronics, in accordance with certain aspects of the present disclosure. For example, system 104 may be configured to continuously monitor one or more analytes of a patient, in accordance with certain aspects of the present disclosure.

Analyte sensor system 104 in the illustrated embodiment includes sensor electronics module 138 and one or more continuous analyte sensor(s) 140 (individually referred to herein as continuous analyte sensor 140 and collectively referred to herein as continuous analyte sensors 140) associated with sensor electronics module 138. Sensor electronics module 138 may be in wireless communication (e.g., directly or indirectly) with one or more of display devices 107a, 107b, 107c, and 107d. In certain embodiments, sensor electronics module 138 may also be in wireless communication (e.g., directly or indirectly) with one or more medical devices, such as medical devices 108 (individually referred to herein as medical device 108 and collectively referred to herein as medical devices 108), and/or one or more other non-analyte sensors 142 (individually referred to herein as non-analyte sensor 142 and collectively referred to herein as non-analyte sensor 142).

In certain embodiments, a continuous analyte sensor 140 may comprise one or more sensors for detecting and/or measuring analyte(s). The continuous analyte sensor 140 may be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 140 may be configured to continuously measure analyte levels of a patient using one or more techniques, such as enzymatic techniques, chemical techniques, physical l techniques, electrochemical techniques, spectrophotometric techniques, polarimetric techniques, calorimetric techniques, iontophoretic techniques, radiometric techniques, immunochemical techniques, and the like. The term “continuous,” as used herein, can mean fully continuous, semi-continuous, periodic, etc. In certain aspects, the continuous analyte sensor 140 provides a data stream indicative of the concentration of one or more analytes in the patient. The data stream may include raw data signals, which are then converted into a calibrated and/or filtered data stream used to provide estimated analyte value(s) to the patient.

In certain embodiments, the continuous analyte sensor 140 may be a multi-analyte sensor, configured to continuously measure multiple analytes in a patient's body. For example, in certain embodiments, the continuous multi-analyte sensor 140 may be a single sensor configured to measure lactate, glucose, ketones (e.g., 3-beta-hydroxybutyrate, acetoacetate, acetone, etc.), glycerol, and/or free fatty acids in the patient's body.

In certain embodiments, one or more multi-analyte sensors may be used in combination with one or more single analyte sensors. As an illustrative example, a multi-analyte sensor may be configured to continuously measure lactate and glucose and may, in some cases, be used in combination with an analyte sensor configured to measure only ketones or only potassium. Information from each of the multi-analyte sensor(s) and single analyte sensor(s) may be combined to provide therapy management support using methods described herein. In further embodiments, other non-contact and or periodic or semi-continuous, but temporally limited, measurements for physiological information may be integrated into the system such as by including weight scale information or non-contact heart rate monitoring from a sensor pad under the patient while in a chair or bed, through an infra-red camera detecting temperature and/or blood flow patterns of the patient, and/or through a visual camera with machine vision for height, weight, or other parameter estimation without physical contact.

In certain embodiments, the continuous analyte sensor(s) 140 may comprise a percutaneous wire that has a proximal portion coupled to the sensor electronics module 138 and a distal portion with several electrodes, such as a measurement electrode and a reference electrode. The measurement (or working) electrode may be coated, covered, treated, embedded, etc., with one or more chemical molecules that react with a particular analyte, and the reference electrode may provide a reference electrical voltage. The measurement electrode may generate the analog electrical signal, which is conveyed along a conductor that extends from the measurement electrode to the proximal portion of the percutaneous wire that is coupled to the sensor electronics module 138. After the continuous analyte monitoring system 104 has been applied to epidermis of the patient, continuous analyte sensor(s) 140 penetrates the epidermis, and the distal portion extends into the dermis and/or subcutaneous tissue under epidermis. Other configurations of continuous analyte sensor(s) 140 may also be used, such as a multi-analyte sensor that includes multiple measurement electrodes, each generating an analog electrical signal that represents the concentration levels of a particular analyte.

Generally, a single-analyte sensor generates an analog electrical signal that is proportional to the concentration level of a particular analyte. Similarly, each multi-analyte sensor generates multiple analog electrical signals, and each analog electrical signal is proportional to the concentration level of a particular analyte. As an illustrative example, continuous analyte sensor 140 may include a single-analyte sensor configured to measure lactate concentration levels, and another single-analyte sensor configured to measure glucose concentration levels of the patient. As another illustrative example, continuous analyte sensor(s) 140 may include a single-analyte sensor configured to measure glucose concentration levels, and one or more multi-analyte sensors configured to measure lactate concentration levels, ketone concentration levels, creatinine concentration levels, etc. As yet another illustrative example, continuous analyte sensor(s) 140 may include a multi-analyte sensor configured to measure lactate concentration levels, glucose concentration levels, ketone concentration levels, creatinine concentration levels, etc. Accordingly, continuous analyte sensor(s) 140 is configured to generate at least one analog electrical signal that is proportional to the concentration level of a particular analyte, and sensor electronics module 138 is configured to convert the analog electrical signal into an analyte sensor count values, calibrate the analyte sensor count values based on the sensitivity profile of the continuous analyte sensor(s) 140 to generate measured analyte concentration levels, and transmit the measured analyte concentration level data, including the measured analyte concentration levels, to a display device, such as display devices 107b, 107c, and/or 107d, via a wireless connection. For example, sensor electronics module 138 may be configured to sample the analog electrical signal at a particular sampling period (or rate), such as every 1 second (1 Hz), 5 seconds, 10 seconds, 30 seconds, 1 minute, 3 minutes, 5 minutes, etc., and to transmit the measured analyte concentration data to the display device at a particular transmission period (or rate), which may be the same as (or longer than) the sampling period, such as every 1 minute (0.016 Hz), 5 minutes, 10 minutes, 30 minutes, at the conclusion of the wear period, etc. Depending on the sampling and transmission periods, the measured analyte concentration data transmitted to the display device include at least one measured analyte concentration level having an associated time tag, sequence number, etc.

In certain embodiments, continuous analyte sensor(s) 140 may incorporate a thermocouple within, or alongside, the percutaneous wire to provide an analog temperature signal to the sensor electronics module 138, which may be used to correct the analog electrical signal or the measured analyte data for temperature. In other embodiments, the thermocouple may be incorporated into the sensor electronics module 138 above the adhesive pad, or, alternatively, the thermocouple may contact the epidermis of the patient through openings in the adhesive pad.

In certain embodiments, the sensor electronics module 138 includes, inter alia, processor 133, storage element or memory 134, wireless transmitter/receiver (transceiver) 136, one or more antennas coupled to wireless transceiver 136, analog electrical signal processing circuitry, analog to-digital (A/D) signal processing circuitry, digital signal processing circuitry, a power source for continuous analyte sensor(s) 140 (such as a potentiostat), etc.

Processor 133 may be a general-purpose or application-specific microprocessor, an application-specific integrated circuit (ASIC), a field programmable gate array (FPGA), etc., that executes instructions to perform control, computation, input/output, etc, functions for the sensor electronics module 138. Processor 133 may include a single integrated circuit, such as a micro processing device, or multiple integrated circuit devices and/or circuit boards working in cooperation to accomplish the appropriate functionality. In certain embodiments, processor 133, memory 134, wireless transceiver 136, the A/D signal processing circuitry, and the digital signal processing circuitry may be combined into a system-on-chip (SoC).

Generally, processor 133 may be configured to sample the analog electrical signal using the A/D signal processing circuitry at regular intervals (such as the sampling instant or period) to generate analyte sensor count values based on the analog electrical signals produced by the continuous analyte sensor(s) 140, calibrate the analyte sensor count values based on the sensitivity profile of the continuous analyte sensor(s) 140 to generate measured analyte concentration levels, and generate measured analyte data from the measured analyte concentration levels, generate sensor data packages that include, inter alia, the measured analyte concentration level data. Processor 133 may store the measured analyte concentration level data in memory 134, and generate the sensor data packages at regular intervals (such as the transmission period) for transmission by wireless transceiver 136 to a display device, such as display devices 107b, 107c, 107d, and/or 107a. Processor 133 may also add additional data to the sensor data packages, such as supplemental sensor information that includes a sensor identifier, a sensor status, temperatures that correspond to the measured analyte data, etc. The sensor data packages are then wirelessly transmitted over a wireless connection to the display device. In certain embodiments, the wireless connection is a Bluetooth or BLE connection. In such embodiments, the sensor data packages are transmitted in the form of Bluetooth or BLE data packets to the display device.

In certain aspects, the transceiver 136 maintains, in the memory 134, a connection list that indicates each device currently connected thereto. In certain aspects, the connection list can also be maintained in other storage locations (e.g., in a cloud computing environment, on each individual display device, etc.). A device may be identified as “currently connected” to the transceiver 136 if the device has successfully engaged in the wireless or wired communication of data (e.g., by requesting and/or receiving at least a predetermined amount of analyte data) with the transceiver 136 via one or more predetermined communication protocols within a predetermined period of time from the current time, has performed and successfully completed one or more wired or wireless transmission security protocols with the transceiver 136 within a predetermined period of time from the current time, etc. In certain aspects, the transceiver 136 can advertise on a regular interval (e.g., every five minutes), such that devices are removed from the connection list after a predetermined number of connections are missed (e.g., one, two, three, etc.). The connection list may include, for each device indicated therein, an identifier of the device (e.g., device identifier, user assigned identifier, transceiver 136 assigned identifier, etc.), a timestamp of last connection, signal strength, an indication of any recent communication errors, and/or other information.

In various embodiments, memory 134 may include volatile and nonvolatile medium. For example, memory 134 may include combinations of random access memory (RAM), dynamic RAM (DRAM), static RAM (SRAM), read only memory (ROM), flash memory, cache memory, and/or any other type of non-transitory computer-readable medium. Memory 134 may store one or more analyte sensor system applications, modules, instruction sets, etc. for execution by processor 133, such as instructions to generate measured analyte data from the analyte sensor count values, etc.

Memory 134 may also store certain sensor operating parameters 135, such as a calibration slope (or calibration sensitivity), a calibration baseline, etc. In particular, the calibration sensitivity, calibration baseline, and other information related to the sensitivity profile for the sensor electronics module 138 may be programmed into the sensor electronics module 138 during the manufacturing process, and then used to convert the analyte sensor electrical signals into measured analyte concentration levels. For example, as discussed above, the calibration slope may be used to predict an initial in vivo sensitivity (M₀) and a final in vivo sensitivity (M_{f}), which are stored in memory 134 and used to convert the analyte sensor electrical signals into measured analyte concentration levels. In certain embodiments, calibration sensitivity (M_{CC}) 146 and/or calibration baseline 147 may be stored in memory 134.

In certain embodiments, sensor electronics module 138 includes electronic circuitry associated with measuring and processing the continuous analyte sensor data, including prospective algorithms associated with processing and calibration of the sensor data. Sensor electronics module 138 can be physically connected to continuous analyte sensor(s) 140 and can be integral with (non-releasably attached to) or releasably attachable to continuous analyte sensor(s) 140. Sensor electronics module 138 may include hardware, firmware, and/or software that enable measurement of levels of analyte(s) via continuous analyte sensor(s) 140. For example, sensor electronics module 138 can include a potentiostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 107b, 107c, 107d, and/or 107a are configured for displaying displayable sensor data, including analyte data, which may be transmitted by sensor electronics module 138. Each of display devices 107b, 107c, 107d, or 107a may include a display such as a touchscreen display 109b, 109c, 109d, and/or 109a for displaying sensor data to a patient or other user and/or for receiving inputs from the patient. For example, a graphical user interface (GUI) may be presented to the patient for such purposes. In certain embodiments, the display devices may include other types of user interfaces such as a voice user interface instead of, or in addition to, a touchscreen display for communicating sensor data to the patient of the display device and/or for receiving patient inputs. Display devices 107a, 107b, 107c, and 107d may be examples of display device 107 illustrated in FIG. 1 used to display sensor data to a patient of the system of FIG. 1 and/or to receive input from the patient.

In certain embodiments, one, some, or all of the display devices are configured to display or otherwise communicate (e.g., verbalize) the sensor data as it is communicated from the sensor electronics module (e.g., in a customized data package that is transmitted to display devices based on their respective preferences), without any additional prospective processing required for calibration and real-time display of the sensor data.

The plurality of display devices may include a custom display device specially designed for displaying certain types of displayable sensor data associated with analyte data received from sensor electronics module. In certain embodiments, the plurality of display devices may be configured for providing alerts/alarms based on the displayable sensor data. Display device 107b is an example of such a custom device. In certain embodiments, one of the plurality of display devices is a smartphone, such as display device 107c which represents a mobile phone, using a commercially available operating system (OS), and configured to display a graphical representation of the continuous sensor data (e.g., including current and historic data). Other display devices can include other hand-held devices, such as display device 107d which represents a tablet, display device 107a which represents a smart watch or fitness tracker, medical device 108 (e.g., an insulin delivery device or a blood glucose meter), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end user, such as the patient) for each particular display device. Accordingly, in certain embodiments, a plurality of different display devices can be in direct wireless communication with a sensor electronics module (e.g., such as an on-skin sensor electronics module 138 that is physically connected to continuous analyte sensor(s) 140) during a sensor session to enable a plurality of different types and/or levels of display and/or functionality associated with the displayable sensor data.

As mentioned, sensor electronics module 138 may be in communication with a medical device 108. Medical device 108 may be a passive device in some example embodiments of the disclosure. For example, medical device 108 may be an insulin pump for administering insulin to a patient. For a variety of reasons, it may be desirable for such an insulin pump to receive and track lactate, glucose, ketones, glycerol and free fatty acid values transmitted from analyte sensor systems 104, where continuous analyte sensor 140 is configured to measure lactate, glucose, ketones, glycerol, and/or free fatty acids.

Further, as mentioned, sensor electronics module 138 may also be in communication with other non-analyte sensors 142. Non-analyte sensors 142 may include, but are not limited to, an altimeter sensor, an accelerometer sensor, a global positioning system (GPS) sensor, a temperature sensor, a respiration rate sensor, etc. Non-analyte sensors 142 may also include monitors such as heart rate monitors, blood pressure monitors, pulse oximeters, caloric intake monitors, indirect calorimetry devices, continuous positive airway pressure machines, and medicament delivery devices. One or more of these non-analyte sensors 142 may provide data to therapy management engine 112 described further below. In some aspects, a patient may manually provide some of the data for processing by the therapy management engine 112 of FIG. 1.

In certain embodiments, non-analyte sensors 142 may further include sensors for measuring movement or activity, skin temperature, core temperature, sweat rate, and/or sweat composition.

In certain embodiments, the non-analyte sensors 142 may be combined in any other configuration, such as, for example, combined with one or more continuous analyte sensors 140. As an illustrative example, a non-analyte sensor, e.g., a temperature sensor, may be combined with a continuous glucose sensor 140 to form a glucose/temperature sensor used to transmit sensor data to the sensor electronics module 138 using common communication circuitry. As another illustrative example, a non-analyte sensor, e.g., a temperature sensor, may be combined with a multi-analyte sensor 140 configured to measure lactate and glucose to form a lactate/glucose/temperature sensor used to transmit sensor data to the sensor electronics module 138 using common communication circuitry.

In certain embodiments, a wireless access point (WAP) may be used to couple one or more of analyte sensor system 104, the plurality of display devices, medical device(s) 108, and/or non-analyte sensor(s) 142 to one another. For example, such WAP may provide Wi-Fi and/or cellular connectivity among these devices. NFC and/or Bluetooth may also be used among devices depicted in diagram 150 of FIG. 1B.

FIG. 2 illustrates example inputs and example metrics that are generated based on the inputs in accordance with certain embodiments of the disclosure. In particular, FIG. 2 illustrates example inputs 127 on the left, application 106 and therapy management engine 112, with DAM 111, in the middle, and example outputs 130 on the right. In certain embodiments, application 106 may obtain inputs 127, in the form of time-series data, through one or more channels (e.g., continuous analyte sensor(s) 140, non-analyte sensor(s) 142, various applications executing on display device 107, etc.). Inputs 127 may be further processed by DAM 111 to output a plurality of metrics, such as outputs 130. Further, inputs (e.g., inputs 127) and metrics (e.g., outputs 130) may be used by the DAM 111 and/or any computing device in the system 100 to perform various processes. Any of inputs 127 may be used for computing any of outputs 130. In certain embodiments, each one of outputs 130 may correspond to one or more values, e.g., discrete numerical values, ranges, or qualitative values (high/medium/low or stable/unstable). In some embodiments, some or all of outputs 130 may include time-series data and/or be provided in the form of time-series data.

In certain embodiments, inputs 127 include food consumption information. Food consumption information may include information about one or more of meals, snacks, and/or beverages, such as one or more of the size, content (carbohydrate, fat, protein, etc.), sequence of consumption, and time of consumption. In certain embodiments, food consumption may be provided by the user through manual entry, by providing a photograph through an application that is configured to recognize food types and quantities, and/or by scanning a bar code or menu. In various examples, meal size may be manually entered as one or more of calories, quantity (e.g., ‘three cookies’), menu items (e.g., ‘Royale with Cheese’), and/or food exchanges (1 fruit, 1 dairy). In some examples, meals may also be entered with the user's typical items or combinations for this time or setting (e.g., workday breakfast at home, weekend brunch at restaurant). In some examples, meal information may be received via a convenient user interface provided by application 106.

In certain embodiments, inputs 127 include activity information. Activity information may be provided, for example, the one or more non-analyte sensors 142 of FIG. 1B. In certain embodiments, activity information may additionally be provided through manual input by user 102. Activity information may include, for example, a time series for each of heart rate, activity minutes, step count, floors climbed, location information (e.g., GPS data), calories burned, sleep duration and/or quality, activity level (e.g., light, medium, or heavy), and/or similar information. In addition, or alternatively, the activity information can include one or more time series for recorded activities of one or more defined activity types (e.g., walk, run, sprint, swim, weightlift etc.), where each activity is associated with a duration and/or time period.

In certain embodiments, inputs 127 include patient statistics, such as one or more of age, height, weight, body mass index, body composition (e.g., % body fat), stature, build, or other information. Patient statistics may be provided through a user interface, by interfacing with an electronic source such as an electronic medical record, and/or from measurement devices. The measurement devices may include one or more of a wireless, e.g., Bluetooth-enabled, weight scale and/or camera, which may, for example, communicate with the display device 107 to provide patient data.

In certain embodiments, inputs 127 include information relating to the user's medication intake. For example, the user's medication intake may include the user's insulin delivery. Such information may be received, via a wireless connection on a smart pen, via user input, and/or from an insulin pump (e.g., medical device 108). Insulin delivery information may include one or more of insulin volume, time of delivery, etc. Other configurations, such as insulin action time or duration of insulin action, may also be received as inputs.

In certain embodiments, inputs 127 include physiological information received from non-analyte sensor(s) 142, which may detect one or more of heart rate, respiration, oxygen saturation, body temperature, etc. (e.g., to detect illness, stress levels, etc.). In certain embodiments, inputs 127 include time, such as time of day, or time from a real-time clock.

In certain embodiments, inputs 127 include analyte data, which may be provided as input from analyte sensor system 104, for example, in any of the ways described with respect to FIG. 1A. An example of analyte data is glucose data, which may be provided and/or stored as a time series corresponding to time-stamped glucose measurements over time. Other types of analyte data, such as ketone data, potassium data, lactate data, etc., may similarly be provided and/or stored as a time series.

As described above, in certain embodiments, DAM 111 generates, determines, and/or computes outputs 130 based on inputs 127 associated with user 102. An example list of outputs 130 is illustrated in FIG. 2. In certain embodiments, outputs 130 generated, determined, or computed by DAM 111 include metabolic rate. Metabolic rate is a metric that may indicate or include a basal metabolic rate (e.g., energy consumed at rest) and/or an active metabolism, e.g., energy consumed by activity, such as exercise or exertion. In some examples, basal metabolic rate and active metabolism may be tracked as separate metric. In certain embodiments, the metabolic rate may be calculated by DAM 111 based on one or more of inputs 127, such as one or more of activity information, sensor input, time, user input, etc.

In certain embodiments, outputs 130 generated, determined, or computed by DAM 111 include an activity level metric. The activity level metric may indicate a level of activity of the user. In certain embodiments, the activity level metric may be determined, for example based on input from an activity sensor or other physiologic sensors. In certain embodiments, the activity level metric may be calculated by DAM 111 based on one or more of inputs 127, such as one or more of activity information, physiological information, analyte data, time, user input, etc. Activity level may indicate whether the user is exercising, at rest, sleeping, etc.

In certain embodiments, outputs 130 generated, determined, or computed by DAM 111 include an insulin resistance metric (also referred to herein as an “insulin resistance”). The insulin resistance metric may be determined using historical data, real-time data, or a combination thereof, and may, for example, be based upon one or more inputs 127, such as one or more of food consumption information, blood glucose information, insulin delivery information, the resulting glucose levels, etc. In certain embodiments, the insulin on board metric may be determined using insulin delivery information, and/or known or learned (e.g., from patient data) insulin time action profiles, which may account for both basal metabolic rate (e.g., update of insulin to maintain operation of the body) and insulin usage driven by activity or food consumption.

In certain embodiments, outputs 130 generated, determined, or computed by DAM 111 include a meal state metric. The meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first.).

In certain embodiments, outputs 130 generated, determined, or computed by DAM 111 include health and sickness metrics. Health and sickness metrics may be determined, for example, based on one or more of user input (e.g., pregnancy information or known sickness information), from non-analyte sensor(s) 142, such as physiologic sensors (e.g., temperature), activity sensors, or a combination thereof. In certain embodiments, based on the values of the health and sickness metrics, for example, the user's state may be defined as being one or more of healthy, ill, rested, or exhausted. In certain embodiments, health and sickness metric may indicate the user's heart rate, stress level, etc.

In certain embodiments, outputs 130 generated, determined, or computed by DAM 111 include analyte level metrics. Analyte level metrics may be determined from analyte data (e.g., glucose measurements obtained from analyte sensor system 104). In some examples, an analyte level metric may also be determined, for example, based upon historical information about analyte levels in particular situations, e.g., given a combination of food consumption, insulin, and/or activity. An analyte level metric may include a rate of change of the analyte, time in range, time spent below a threshold level, time spent above a threshold level, or the like. In certain embodiments, an analyte trend may be determined based on the analyte level over a certain period of time. As described above, example analytes may include glucose, ketones, lactate, potassium and others described herein.

In certain embodiments, outputs 130 generated, determined, or computed by DAM 111 include a disease stage. For example disease stages for Type II diabetics may include a pre-diabetic stage, an oral treatment stage, and a basal insulin treatment stage. In certain embodiments, degree of glycemic control (not shown) may also be determined as an outcome metric, and may be based, for example, on one or more of glucose levels, variation in glucose level, or insulin dosing patterns.

In certain embodiments, outputs 130 generated, determined, or computed by DAM 111 include clinical metrics. Clinical metrics generally indicate a clinical state a user is in with respect to one or more conditions of the user, such as diabetes. For example, in the case of diabetes, clinical metrics may be determined based on glycemic measurements, including one or more of A1C, trends in A1C, time in range, time spent below a threshold level, time spent above a threshold level, and/or other metrics derived from glucose values. In certain embodiments, clinical metrics may also include one or more of estimated A1C, glycemic variability, hypoglycemia, and/or health indicator (time magnitude out of target zone).

FIG. 3 illustrates an example of a system 300 for sharing a patient's health data between devices and/or with one or more other users (referred to herein as supporters), in accordance with certain embodiments. System 300 includes the analyte sensor system 104 of FIGS. 1A-B, the display devices 107a and 107c of FIG. 1B, a supporter device 311, a mesh device 360, and a server system 330. The display devices 107a and 107c, the supporter device 311, and the server system 330 are interconnected via a network 316. The network 316 can be a local area network (LAN), wide area network (WAN), a cellular network, the Internet, or any other group of connected computing devices. Although the mesh device 360 is illustrated singly for simplicity, the mesh device 360 is representative of a variety of devices capable of communicating via a wireless (e.g., Bluetooth, Wi-Fi) or wired communication protocol (e.g., Ethernet). Examples of mesh devices 360 can include, but are not limited to, an insulin pump, smart thermostat, smart speaker, smart appliance, router, access point, etc. In addition, or alternatively, in some aspects, the mesh devices 360 can include display devices such as the display devices 107a and/or 107c of FIG. 1B.

The user 102 can use one or both of the display devices 107a and 107c to collect sensor data and monitor their health. More particularly, in the illustration of FIG. 3, the display devices 107a and 107c are examples of multiple receiver devices, namely, a smartphone and a smartwatch, that each directly communicate with, and obtain sensor data from, the analyte sensor system 104 worn by the user 102, where such sensor data can be transmitted by the analyte sensor system 104 in the fashion described relative to FIGS. 1A-B. According to the example of FIG. 3, the display devices 107a and 107c may each be included in the connection list discussed relative to the transceiver 136 of FIG. 1B. Although two receiver devices are shown in FIG. 3, it should be appreciated that, in various implementations, any number of devices may directly receive such sensor data including, for example, any one or more of display devices 107a, 107b, 107c, and 107d of FIG. 1B. Display devices 107a and 107c may include touchscreen displays 109a and 109c, respectively, as discussed relative to FIG. 1B. As shown, display devices 107a and 107c execute application 106-1 and application 106-2, respectively, which applications each correspond to instances of the application 106 discussed relative to FIGS. 1A-B and 2.

In certain aspects, a supporter 308 uses the supporter device 311 to help the user 102 manage their health or medical condition, such as diabetes. The supporter device 311 can execute an application 306, which can access at least selected health data of the user 102 (e.g., analyte data and/or alerts related thereto) from the server system 330 and can display the health data via a display 309 of the supporter device 311. In some aspects, the application 306 can operate as described relative to the application 106 of FIG. 1. Although the supporter 308 and the supporter device 311 are shown singly in FIG. 3, it should be appreciated that, in various implementations, the supporter 308 and/or the supporter device 311 may be representative of multiple supporters and/or supporter devices, respectively.

In the example of FIG. 3, the server system 330 is an example of a remote target that facilitates, for example, publishing or providing health data of the user 102 to the supporter 308. In some aspects, the server system 330 is operable in a cloud computing environment (e.g., a private or public cloud) and can provide application services and/or features for the analyte sensor system 104 and/or applications 106-1, 106-2, and/or 306. In certain aspects, the server system 330 implements at least a portion of the therapy management engine 112 described with respect to FIG. 1A. The server system 330 can receive and store data generated by the analyte sensor system 104 (via transmissions initiated by the applications 106-1 and/or 106-2), monitor operating parameters of the analyte sensor system 104 (via the applications 106-1 and/or 106-2), publish or provide health data to the supporter device 311, push updates to the applications 106-1, 106-2 and/or 306, etc.

The server system 330 includes a processor 331 configured to execute one or more software applications stored in a memory 332. For example, the processor 331 can execute a data sharing application generally configured to enable the supporter 308 to receive health data of the user 102 and monitor the health of the user 102. For example, in some aspects, once the user 102 opts into receiving services provided by the server system 330, the server system 330 is able to receive the health data transmitted by the display devices 107a and/or 107c and to store the health data in a storage system (e.g., data store 310). Once the user 102 opts into allowing the supporter 308 to receive information related to the health of the user 102, the server system 330 can allow the supporter 308 to also access the health data within the storage system.

In general, to enable publication of health data of the user 102 to the supporter 308, the analyte sensor system 104 may be configured to generate time-series sensor data, such as analyte measurements, for the user 102, e.g., on a continuous basis. The analyte sensor system 104 transmits the sensor data to each receiver device connected thereto (e.g., any of the display devices 107) via, for example, the transceiver 136 discussed relative to FIG. 1B. For example, the analyte sensor system 104 can transmit the sensor data to each device included in the connection list discussed relative to the transceiver 136 of FIG. 1B. In the example of FIG. 3, the sensor data may be transmitted to each of the display devices 107a and 107c for use by the application 106-1 and the application 106-2, respectively.

In certain aspects, one or both of the display device 107a and the display device 107c may transmit the sensor data to the server system 330. The server system 330 can forward the sensor data and/or alerts or other health data based on the sensor data to the supporter device 311, thereby allowing the supporter 308 to view the patient's analyte data and/or alerts substantially in real-time. In some aspects, any resulting redundant transmissions to the server system 330 can be de-duplicated upon receipt of the sensor data, upon transmission of alerts, for example, to the supporter device 311, and/or the like. The redundant transmissions can be de-duplicated, for example, based on timestamps, a combination of timestamps and data content, and/or the like.

In the example of FIG. 3, the display devices 107a and 107c are configured to directly communicate with the analyte sensor system 104, as discussed above, and also to directly communicate with each other. In particular, communication paths are established between the analyte sensor system 104 and the display device 107a, between the analyte sensor system 104 and the display device 107c, and between the display device 107a and the display device 107c. In certain aspects, communication between the display device 107a and the display device 107c, for example, can occur over the network 316. In addition, or alternatively, such communication can occur directly between the two devices without the network 316.

In some cases, as discussed previously, the display device 107a and/or the display device 107c may periodically experience a communication disruption with the analyte sensor system 104, thus impacting that device's access to analyte data. In certain aspects, the three communication paths discussed above may form a “triangle of success” so as to mitigate such communication disruptions. Although certain illustrative examples are described herein in terms of two devices and an analyte sensor forming a “triangle of success,” it should appreciated that similar reliability and redundancy can be achieved with four, five, six, or any other suitable number devices that are each operable to communicate with an analyte sensor and each other. For example, in some aspects, the mesh device 360 can provide a protocol stack for enabling additional communication paths via, for example, a mesh network.

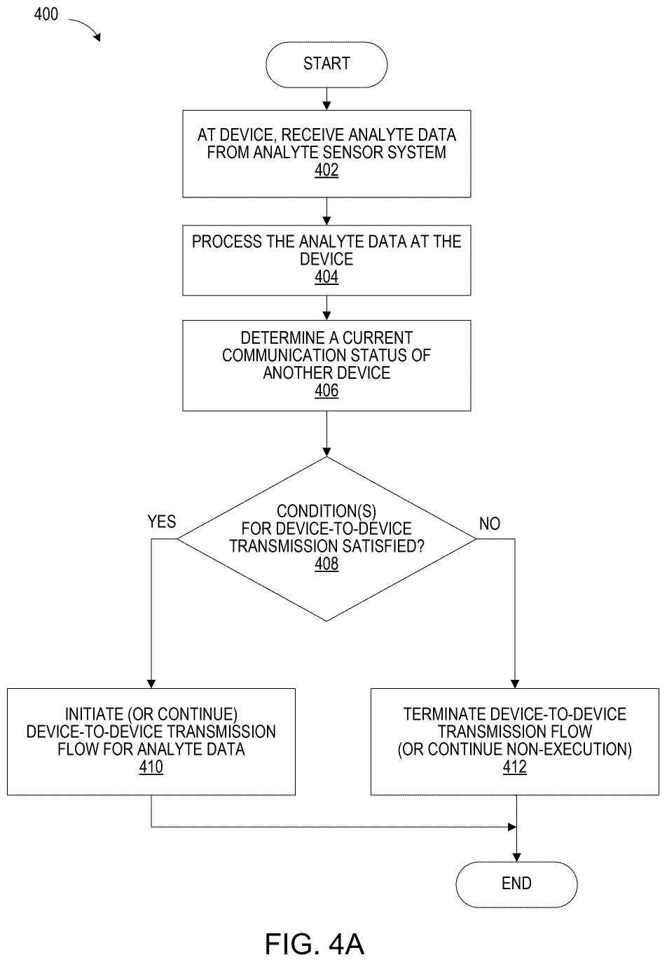

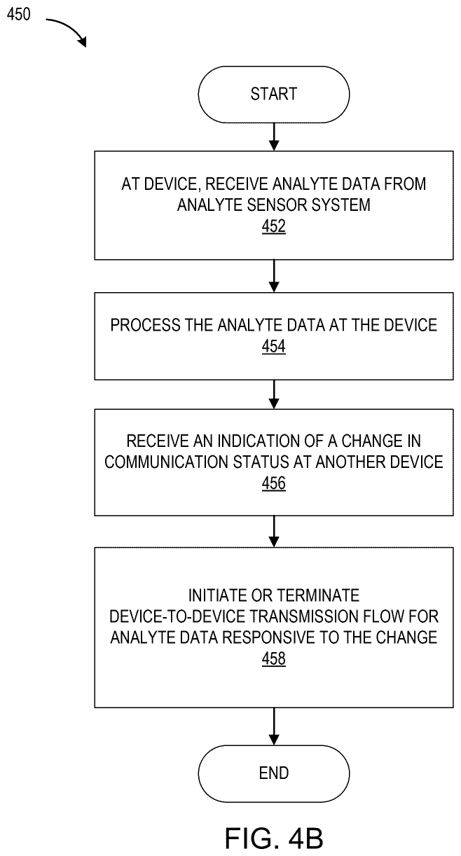

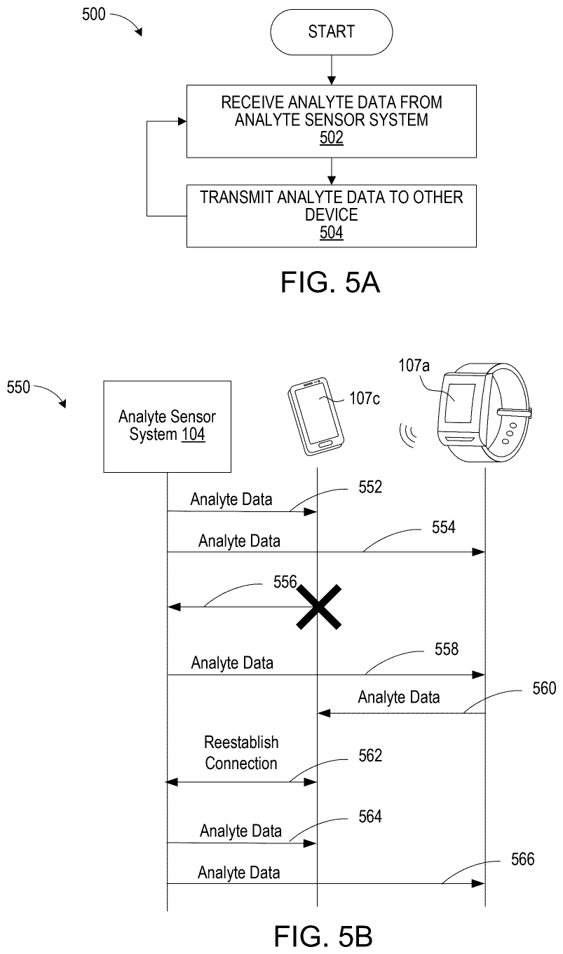

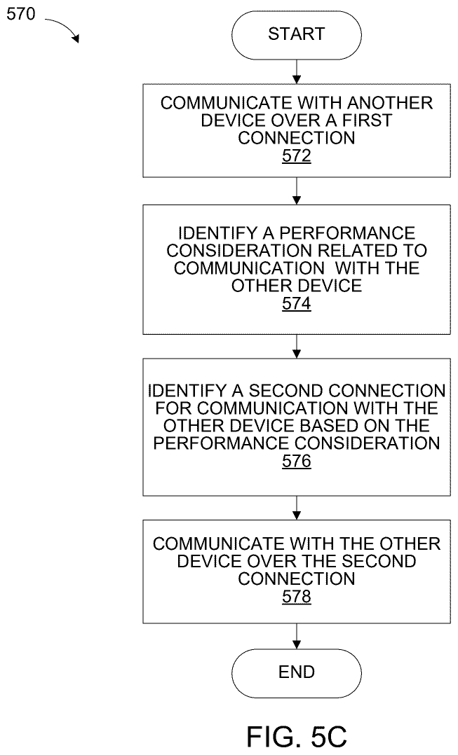

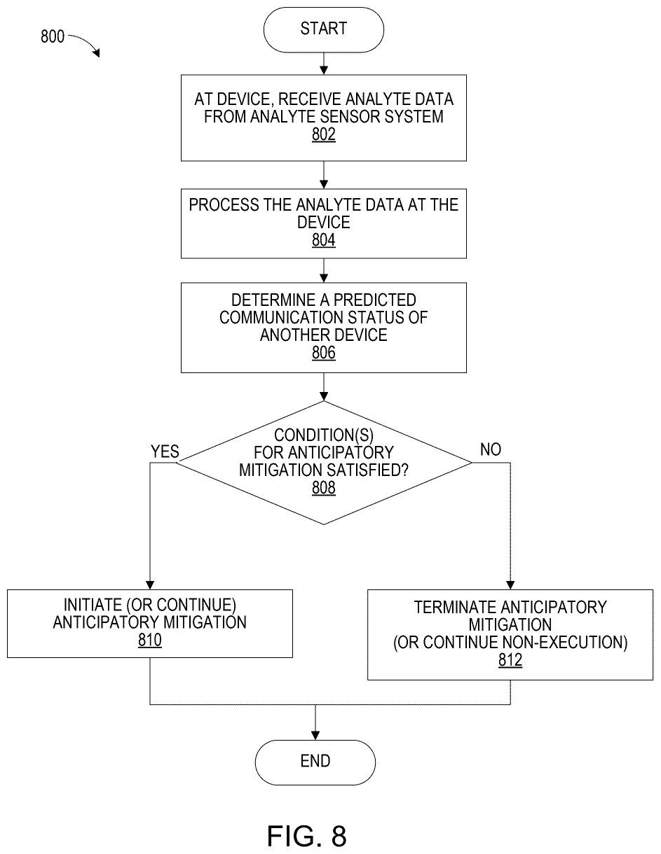

In certain aspects, the display devices 107a and 107c can each be configured to serve as an alternative source of health data, such as analyte data, in the event the other device experiences a communication disruption with the analyte sensor system 104. In certain aspects, the applications 106-1 and 106-2 can each be configured to execute a process for device-to-device mitigation of disrupted communication with analyte sensors. For example, the application 106-1 on the display device 107a can initiate a device-to-device transmission flow if the application 106-1 determines, for example, that the display device 107c is no longer connected to the analyte sensor system 104. According to this example, the device-to-device transmission flow can facilitate, for example, transfer of all or a portion of analyte data received by the display device 107a to the display device 107c. Examples of processes for device-to-device mitigation will be discussed relative to FIGS. 4A-B. Examples of processes for executing device-to-device transmission flows will be described relative to FIGS. 5A-C, 6A-B, and 7A-B. An example of a process for anticipatory mitigation of a predicted communication disruption will be described relative to FIG. 8.

FIG. 4A illustrates an example of a process 400 for device-to-device mitigation of disrupted communication with analyte sensors, in accordance with certain embodiments. In some embodiments, the process 400 can be executed, for example, by the therapy management engine 112 of FIGS. 1A-B and 2. In addition, or alternatively, the process 400 can be executed, for example, by the application 106 of FIGS. 1A-B and 2. In addition, or alternatively, the process 400 can be executed generally by any of the display devices 107 of FIGS. 1A-B and 2.

Although any number of systems, in whole or in part, can implement the process 400, to simplify discussion, the process 400 will be described as being performed by the application 106 of FIGS. 1A-B and 2, with particular reference to the system 300 of FIG. 3. With reference to the system 300 of FIG. 3, in some aspects, the process 400 can be executed continuously by each of the applications 106-1 and 106-2. In addition, or alternatively, the process 400 can be executed by the application 106-1 and the application 106-2 at any suitable interval (e.g. every 5 minutes, every 10 minutes, every 15 minutes, etc.). Other variations will be apparent to one skilled in the art after a detailed review of the present disclosure.

At block 402, at the display device 107, the application 106 receives analyte data (e.g., analyte measurements) from the analyte sensor system 104. The analyte data may be received, for example, via the transceiver 136 of the analyte sensor system 104, as discussed relative to FIGS. 1A-B and 3.

At block 404, the application 106 processes the analyte data at the display device 107. In general, the block 404 can include the application 106 executing logic to store, analyze and/or present (e.g., display) the analyte data according to its standard application flow. In addition, or alternatively, the block 404 can include, for example, generating and/or presenting alerts based on the analyte data (e.g., high, low, rapidly increasing, rapidly decreasing, etc.).

At block 406, the application 106 determines a current communication status of another device. In general, the current communication status characterizes a current ability of the other device to communicate with the analyte sensor system 104 and/or the display device 107. In an example, if the process 400 is performed by the application 106-1 executing on the display device 107a, the block 406 can include determining a current communication status of the display device 107c. In another example, if the process 400 is performed by the application 106-2 executing on the display device 107c, the block 406 can include determining a current communication status of the display device 107a.

In certain aspects, the current communication status can characterize a connection between the other device and the analyte sensor system 104. The current communication status can include, for example, an indication of whether the other device is currently connected to the analyte sensor system 104. In an example, the application 106 can retrieve a connection list for the analyte sensor system 104, such as the connection list discussed relative to the transceiver 136 of FIG. 1B. According to this example, the connection list can be retrieved from the transceiver 136 or from another storage location where the connection list may be maintained, such as the server system 330 and/or a storage location on the display device 107 or another device. As discussed previously, the connection list may indicate, for example, each device currently connected to the analyte sensor system 104. According to this example, if the other device is not included or indicated in the connection list, it may be determined that that the other device is not currently connected to the analyte sensor system 104.

In another example utilizing a connection list, as discussed previously, the connection list can include a timestamp of a last connection between the analyte sensor system 104 and the other device. According to this example, the application 106 can determine, from the timestamp in the connection list, an elapsed time since the last connection between the analyte sensor system 104 and the other device. In certain aspects, the elapsed time can be used at decision block 408 (discussed below) to determine that the analyte sensor system 104 has not communicated with the other device within a specified period (e.g., five minutes). The specified period can be, for example, a shorter period than is necessary for the other device to be removed from the connection list. Therefore, according to this example, the elapsed time analysis enables a connection interruption to be identified and responded to more quickly and efficiently. In addition, or alternatively, the other device can notify the display device 107c that it is no longer connected to the analyte sensor system 104. In addition, or alternatively, the analyte sensor system 104 can notify the display device 107 that the other device is no longer connected.

In certain aspects, the current communication status can characterize a connection between the other device and the display device 107. The current communication status can include, for example, an indication of whether the other device is currently connected to the display device 107. In some aspects, the application 106 can determine whether the other device is currently connected to the display device 107, for example, by causing the display device 107 to periodically ping the other device.

In certain aspects, the current communication status can include, for example, dynamic attributes of the other device, the connection between the other device and the analyte sensor system 104, and/or the connection between the other device and the display device 107. The dynamic attributes can include, for example, a signal strength associated with the other device's connection with the analyte sensor system 104, a signal strength associated with the other device's connection with the display device 107, a power level (e.g., battery level) of the other device, an indication of any communication errors with the other device, an indication of any technical problems on the other device (e.g., current inability to present local notifications due to a battery level or a device setting, a pending shutdown or restart, etc.), combinations of the foregoing and/or the like. The dynamic attributes can be received or retrieved, for example, from the analyte sensor system 104 (e.g., via the connection list described above) and/or from the other device.

At decision block 408, the application 106 determines, based on the current communication status of the other device, whether one or more conditions for device-to-device transmission, or side-filling, are satisfied. In an example, the condition(s) can be satisfied if the current communication status indicates that the other device is not currently connected to the analyte sensor system 104. In another example, the condition(s) can be satisfied if the current communication status indicates that that the power level (e.g., battery level) of the other device is below a threshold. In another example, the condition(s) can be satisfied if the current communication status indicates that that the signal strength of the other device's connection with the analyte sensor system 104 is below a threshold. In another example, the condition(s) can be satisfied if the current communication status indicates a threshold number of communication errors within a specified period. In another example, the condition(s) can be satisfied if the current communication status indicates no communication between the analyte sensor system 104 and the other device within a specified period (e.g., five minutes). Other examples will be apparent to one skilled in the art after a detailed review of the present disclosure.

If it is determined, at the decision block 408, that the conditions(s) for device-to-device transmission are satisfied, at block 410, the application 106 initiates a device-to-device transmission flow for analyte data. In some cases, the initiating can involve updating a corresponding setting, for example, by turning the setting to “ON.” In some aspects, if the device-to-device transmission flow is already initiated, for example, due to a command or a previous iteration of the process 400, the device-to-device transmission flow is allowed to continue execution.

In general, the device-to-device transmission flow can involve the display device 107 facilitating transfer of all or a portion of received analyte data (e.g., analyte measurements) to the other device. For example, the display device 107 can take action to transmit analyte data to the other device and/or cache analyte data for later transmission to the other device. The display device 107 can thereby serve as an alternative source of analyte data for the other device. In some aspects, the device-to-device transmission flow can involve temporarily changing operation of the analyte sensor system 104, such as by causing the transceiver 136 to temporarily boost power, for example, to accommodate a less sensitive transceiver of the display device 107 as compared to the other device, if applicable (e.g., +4 dB, if the display device 107 is smartwatch rather than a smartphone). In certain aspects, the device-to-device transmission flow can continue to execute until the condition(s) for device-to-device transmission are no longer satisfied.

In some aspects, the application 106 can initiate different device-to-device transmission flows for different communication statuses. For example, if the other device is not currently connected to the analyte sensor system 104, but is currently connected to the display device 107, the application 106 can initiate a flow in which the display device 107 forwards analyte data to the other device in real-time. In another example, if the other device is not currently connected to either the analyte sensor system 104 or the display device 107, the application 106 can initiate a flow in which the display device 107 caches analyte data for later transmission to the other device.

In addition, or alternatively, the initiation of the device-to-device transmission flow can involve terminating one device-to-device transmission flow and starting another. For example, if, according to a currently executing flow, the display device 107 is forwarding analyte data to the other device in real-time, and the current communication status indicates that the other device is no longer connected to the display device 107, the application 106 can terminate the currently executing flow and initiate a flow in which the display device 107 caches analyte data for later transmission to the other device. Examples of device-to-device transmission flows will be described in greater detail relative to FIGS. 5A-C, 6A-B, and 7A-B.

If it is determined, at the decision block 408, that the conditions(s) for device-to-device transmission are not satisfied, at block 412, the application 106 terminates the device-to-device transmission flow, if the flow is active or executing. If the device-to-device transmission flow is not active or executing, the non-execution is allowed to continue. In certain aspects, termination of the device-to-device transmission flow can involve updating a setting, for example, by turning the setting to “OFF.” In addition, or alternatively, if operation of the analyte sensor system 104 was temporarily changed at the block 410 (e.g., by boosting power of the transceiver 136), the termination can involve restoring previous or standard operation of the analyte sensor system 104. After either block 410 or 412, the process 400 ends.

FIG. 4B illustrates an example of a process 450 for device-to-device mitigation of disrupted communication with analyte sensors, in accordance with certain embodiments. In some embodiments, the process 450 can be executed, for example, by the therapy management engine 112 of FIGS. 1A-B and 2. In addition, or alternatively, the process 450 can be executed, for example, by the application 106 of FIGS. 1A-B and 2. In addition, or alternatively, the process 450 can be executed generally by any of the display devices 107 of FIGS. 1A-B and 2.

Although any number of systems, in whole or in part, can implement the process 450, to simplify discussion, the process 450 will be described as being performed by the application 106 of FIGS. 1A-B and 2, with particular reference to the system 300 of FIG. 3. With reference to the system 300 of FIG. 3, in some aspects, the process 450 can be executed continuously by each of the applications 106-1 and 106-2. In addition, or alternatively, the process 450 can be executed by the application 106-1 and the application 106-2 at any suitable interval (e.g. every 5 minutes, every 10 minutes, every 15 minutes, etc.). Other variations will be apparent to one skilled in the art after a detailed review of the present disclosure.

At block 452, at the display device 107, the application 106 receives analyte data (e.g., analyte measurements) from the analyte sensor system 104, as discussed relative to the block 402 of FIG. 4A. At block 454, the application 106 processes the analyte data at the display device 107, as discussed relative to the block 404 of FIG. 4A.

At block 456, the application 106 receives an indication of a change in communication status of another device, such as an indication of a communication disruption with the analyte sensor system 104 and/or the display device 107. The indication can be, for example, an indication that the other device is no longer connected to the analyte sensor system 104, that the other device is no longer connected to the display device 107, that the other device has a poor connection with the analyte sensor system 104 (e.g., signal strength below a threshold), that the other device has a poor connection with the display device 107 (e.g., signal strength below a threshold), and/or the like. The indication can be received, for example, from the other device (e.g., the display device 107a or the display device 107c), the mesh device 360, the supporter device 311, the server system 330, and/or the like. In certain aspects, the change in communication status can be associated with a particular device-to-device transmission flow.

In some aspects, the indication received at the block 456 can be a command to initiate or terminate a device-to-device transmission flow. In some aspects, the command be a user command, for example, from the user 102 or the supporter 308. In certain aspects, if the command is to initiate a device-to-device transmission flow, the command can specify or be associated with a termination condition, such as a period of time (e.g., 30 minutes, 2 hours, etc.), a device location (e.g., while the display device 107a is at home, away from home, at a pool, etc.). Accordingly, the command can remain in effect until the termination condition is satisfied. Other examples of triggers for initiating and/or terminating the device-to-device transmission flow will be apparent to one skilled in the art after a detailed review of the present disclosure.

At block 458, the application 106 initiates or terminates a device-to-device transmission flow for analyte data according to the change in communication status at the other device. The block 458 can include, for example, executing any of the functionality described relative to the blocks 410 and 412 of FIG. 4A. After block 458, the process 450 ends.

FIG. 5A illustrates an example of a process 500 for executing a device-to-device transmission flow, in accordance with certain embodiments. In some embodiments, the process 500 can be executed, for example, by the therapy management engine 112 of FIGS. 1A-B and 2. In addition, or alternatively, the process 500 can be executed, for example, by the application 106 of FIGS. 1A-B and 2. In addition, or alternatively, the process 500 can be executed generally by any of the display devices 107 of FIGS. 1A-B and 2. Although any number of systems, in whole or in part, can implement the process 500, to simplify discussion, the process 500 will be described as being performed by the application 106 of FIGS. 1A-B and 2, with particular reference to the system 300 of FIG. 3.

In certain aspects, the process 500 can be initiated as part of the process 400 of FIG. 4A, for example, if a current communication status of another device indicates that the other device is not currently connected to the analyte sensor system 104 and also that the other device is currently connected to the display device 107. In addition, or alternatively, the process 500 can be initiated as part of the process 450 of FIG. 4B, for example, in response to a received indication of a change in communication status of the other device (e.g., a command). In some aspects, the process 500 can execute at the display device 107 in parallel to repeated, or continuous, execution of the process 400 of FIG. 4A.

At block 502, the application 106 receives analyte data from the analyte sensor system 104, in similar fashion to the block 402 of FIG. 4A. In general, the block 502 is illustrative of analyte data to be processed for device-to-device transmission according to the process 500 of FIG. 5A. In certain aspects, the block 502 is a reference to the receipt of analyte data according to the block 402 of FIG. 4A or the block 452 of FIG. 4B. In certain aspects, the application 106 can cache or buffer the analyte data on the display device 107, for example, until block 504 is executed, and remove the analyte data from the buffer or cache after transmitting the analyte data to the other device (e.g., after block 504 is executed).

At block 504, the application 106 causes the display device 107 to transmit the analyte data to the other device, for example, in real-time as the analyte data is received from the analyte sensor system 104. From block 504, the process 500 returns to the block 502 and executes as described previously. In various aspects, the process 500 can continue until conditions for device-to-device transmission are no longer satisfied, for example, according to the process 400 of FIG. 4A. In addition, or alternatively, the process 500 can continue until a command to terminate the process 500 is received, for example, according to the process 450 of FIG. 4B.

FIG. 5B is a sequence diagram 550 illustrating example interactions between the analyte sensor system 104, the display device 107c, and the display device 107a, in accordance with certain embodiments. The sequence diagram 550 will be described in relation to the process 400 of FIG. 4A, the process 450 of FIG. 4B, and the process 500 of FIG. 5A.

Steps 552 and 554 correspond to a period before initiation of a device-to-device transmission flow, such as the process 500 of FIG. 5A. At steps 552 and 554, the analyte sensor system 104 transmits analyte data directly to the display device 107c and the display device 107a, respectively. Concurrent with the steps 552 and 554, the display devices 107a and 107c may each continuously execute a process for device-to-device mitigation of disrupted communication with analyte sensors, such as the process 400 of FIG. 4A discussed above. In addition, or alternatively, the display devices 107a and 107c may each execute a process for device-to-device mitigation based on received indications of communication disruptions (e.g., commands), such as the process 450 of FIG. 4B discussed above.

At step 556, the display device 107c loses connectivity with the analyte sensor system 104, thus beginning a period of communication disruption with the analyte sensor system 104. Subsequent to the step 556, with reference to the process 400 of FIG. 4A, the display device 107a can determine a current communication status of the display device 107c. Alternatively, subsequent to the step 556, with reference to the process 450 of FIG. 4B, the display device 107a can receive an indication of a change in communication status of the display device 107c. According to the example of FIG. 5B, the current communication status, or the received indication of a change in communication status, may indicate that the display device 107c is: a) not currently connected to the analyte sensor system 104; and b) currently connected to the display device 107a. As discussed previously, the received indication of a change in communication status may be, for example, a command to initiate a device-to-device transmission flow, such as the process 500 of FIG. 5A. In response to the current communication status of the display device 107c, or the received indication of the change in communication status, the display device 107a can initiate the process 500 of FIG. 5A.

Steps 558, 560, and 562 correspond to a period during execution of the process 500 of FIG. 5A. At step 558, the analyte sensor system 104 transmits analyte data directly to the display device 107a, in similar fashion to the step 554 discussed above. At step 560, the display device 107a transmits the analyte data to the display device 107c, as discussed relative to the block 504 of FIG. 5A. Steps 558 and 560 can repeat until conditions for device-to-device transmission are no longer satisfied, or until a termination command is received, as discussed previously.

At step 562, the display device 107c reestablishes a connection with the analyte sensor system 104. Subsequent to the step 562, with reference to the process 400 of FIG. 4A, the display device 107a can determine a current communication status of the display device 107c. Alternatively, subsequent to the step 562, with reference to the process 450 of FIG. 4B, the display device 107a can receive an indication of a change in communication status of the display device 107c. According to the example of FIG. 5B, the current communication status, or the received indication of a change in communication status, may indicate that the display device 107c is once again connected to the analyte sensor system 104. As discussed previously, the received indication of a change in communication status may be, for example, a command to terminate the device-to-device transmission flow, such as the process 500 of FIG. 5A. In response to the current communication status of the display device 107c, or the received indication of the change in communication status, the display device 107a can terminate the process 500 of FIG. 5A.

Steps 564 and 566 correspond to a period after termination of a device-to-device transmission flow, such as the process 500 of FIG. 5A. At steps 564 and 566, the analyte sensor system 104 transmits analyte data directly to the display device 107c and the display device 107a, respectively, as discussed relative to the steps 552 and 554.

Advantageously, in certain implementations, the previous execution of the steps 558 and 560 can serve to relieve a resource burden on the analyte sensor system 104. Since, during the communication disruption, the display device 107a transmits analyte data directly to the display device 107c, the analyte sensor system 104 saves the network and processing cost of back-filling analyte data to the display device 107c upon the reestablishment of a connection at the step 562. Further, in certain implementations, the display device 107a can retain real-time access to analyte data, thereby facilitating timely alerting, for example, to the user 102.

FIG. 5C illustrates an example of a process 570 for optimizing communication between devices during execution of a device-to-device transmission flow, such as the process 500 of FIG. 5A, in accordance with certain embodiments. In some embodiments, the process 570 can be executed, for example, by the therapy management engine 112 of FIGS. 1A-B and 2. In addition, or alternatively, the process 570 can be executed, for example, by the application 106 of FIGS. 1A-B and 2. In addition, or alternatively, the process 570 can be executed generally by any of the display devices 107 of FIGS. 1A-B and 2. Although any number of systems, in whole or in part, can implement the process 570, to simplify discussion, the process 570 will be described as being performed by the application 106 of FIGS. 1A-B and 2, with particular reference to the system 300 of FIG. 3.

At block 572, the application 106 causes the display device 107 to communicate with another device over a first or primary connection. The first or primary connection may utilize, for example, BLE communication or another communication method.

At block 574, the application 106 identifies a performance consideration related to the communication of the display device 107 with the other device. In an example, the application 106 can determine a loss of connectivity between the display device 107 and the other device over the first or primary connection (e.g., BLE). In another example, the application 106 can determine that the display device 107 is low on power (e.g., a battery level below a defined threshold). In another example, the application 106 can determine that the display device 107 is in a low power mode such that power efficiency at the display device 107 is prioritized. In another example, the application 106 can identify that a signal strength associated with the other device's connection with the display device 107 is weak (e.g., below a defined threshold).

At block 576, the application 106 identifies a second connection for communication with the other device based on the performance consideration (or a combination of performance considerations). The different or second connection can include, for example, utilization of BLE, cellular, Wi-Fi, satellite, mesh networks, and/or the like. In some aspects, the application 106 can evaluate a plurality of connection options based on the performance consideration. For example, BLE may be associated with low power and low range, Wi-Fi may be associated with medium power and medium range, cellular may be associated with high power and high range, and satellite may be associated with highest power and highest range. It should be appreciated that the foregoing performance associations for different communication protocols are presented solely for illustrative purposes. Other examples of performance associations will be apparent to one skilled in the art after a detailed review of the present disclosure.

In an example using the illustrative performance associations above, if the performance consideration is that the display device 107 is low on power, or that the display device 107 is in a low power mode, the second connection can correspond to a connection option that utilizes less power than the first connection. According to this example, if the first or primary connection utilizes Wi-Fi communication, the second connection can utilize a BLE communication. In another example, if the performance consideration is that the display device 107 has a weak connection with the other device, the second connection can correspond to a connection option that offers more range than the first connection. According to this example, if the first or primary connection utilizes BLE communication, the second connection can utilize Wi-Fi communication.

At block 578, the application 106 causes the display device 107 to communicate with the other device over the second connection. The block 578 can include, for example, establishing the second connection with the other device. After block 578, the process 570 ends.

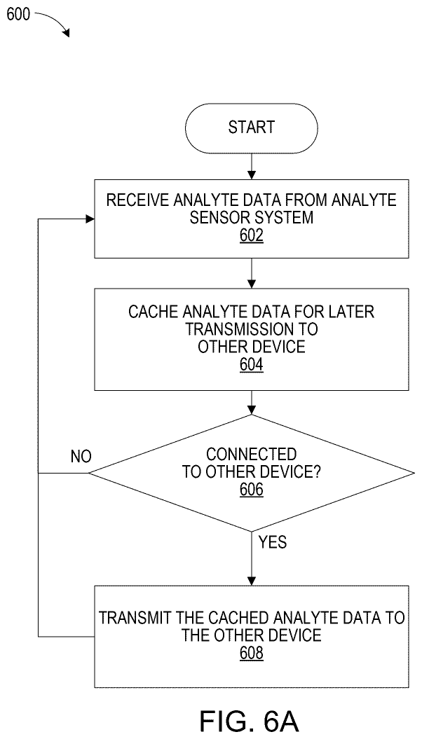

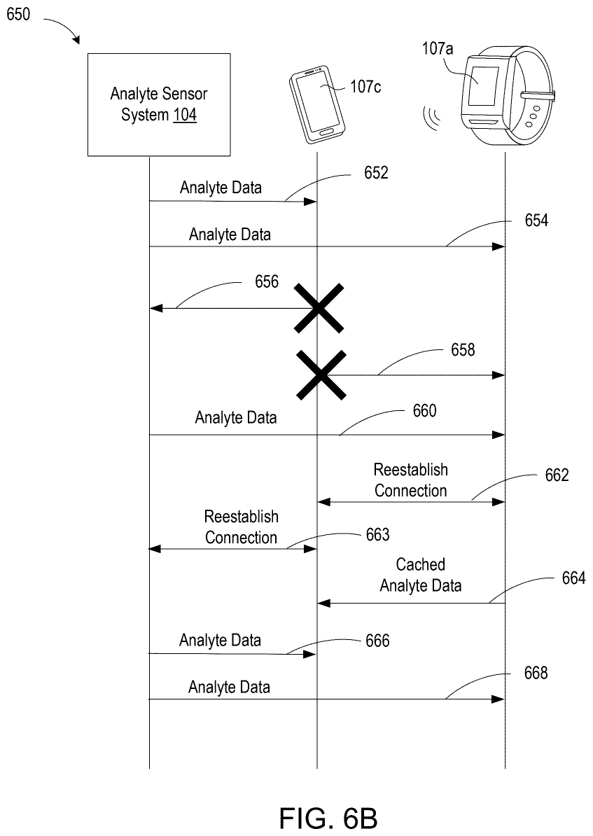

FIG. 6A illustrates an example of a process 600 for executing a device-to-device transmission flow, in accordance with certain embodiments. In some embodiments, the process 600 can be executed, for example, by the therapy management engine 112 of FIGS. 1A-B and 2. In addition, or alternatively, the process 600 can be executed, for example, by the application 106 of FIGS. 1A-B and 2. In addition, or alternatively, the process 600 can be executed generally by any of the display devices 107 of FIGS. 1A-B and 2. Although any number of systems, in whole or in part, can implement the process 600, to simplify discussion, the process 600 will be described as being performed by the application 106 of FIGS. 1A-B and 2, with particular reference to the system 300 of FIG. 3.

In certain aspects, the process 600 can be initiated as part of the process 400 of FIG. 4A, for example, if a current communication status of another device indicates that the other device is not currently connected to either one of the analyte sensor system 104 or the display device 107. In addition, or alternatively, the process 600 can be initiated as part of the process 450 of FIG. 4B, for example, in response to a received indication of a change in communication status of the other device. In various aspects, the process 600 can execute at the display device 107 in parallel to repeated or continuous execution of the process 400 of FIG. 4A.

At block 602, the application 106 receives analyte data from the analyte sensor system 104, in similar fashion to the block 402 of FIG. 4A. In general, the block 602 is illustrative of analyte data to be processed for device-to-device transmission according to the process 600 of FIG. 6A. In certain aspects, the block 602 is a reference to the receipt of analyte data according to the block 402 of FIG. 4A or the block 452 of FIG. 4B.

At block 604, the application 106 causes the analyte data to be cached on the display device 107 for later transmission to the other device. The caching can be implemented, for example, in local storage on the display device 107. In some aspects, the display device 107 can continuously cache the analyte data, even without a disruption, such that the caching continues throughout the period of disruption. In other aspects, the display device 107 can initiate the caching in response to the disruption, as part of the process 600 of FIG. 6A. In still other aspects, as further discussed below, the display device 107 can initiate caching of more or different data than is normally cached by the display device 107.

In certain aspects, as part of the caching at the block 604, the display device 107 can store the same data, or different data, as compared to the analyte sensor system 104. For example, the display device 107a may store data at more frequent time intervals than the analyte sensor system 104, may store data at a greater level of granularity than the analyte sensor system 104, and/or may store additional or different metadata than the analyte sensor system 104 (e.g., location data and/or other obtained metadata). The additional data or metadata can include, for example, timestamps, information related to event identification, information related to alert generation, etc. In some aspects, during the process 600, the analyte sensor system 104 may handoff all or a portion of caching duties to the display device 107, thereby relieving a resource burden on the analyte sensor system 104. In some cases, the handoff can be responsive to the analyte sensor system 104 identifying, for example, that its cache is full, that a predetermined proportion of its cache is full (e.g., 95%), etc. In some aspects, the cached data on the display device can be configurably limited in time (e.g., 24 hours of data, 10 days of data, 15 days of data, etc.).

In some aspects, caching can be configurably distributed between the display device 107a and the analyte sensor system for a duration of the process 600. In an example, the display device 107a may cache some or all of the analyte data and the analyte sensor system 104 may not cache any data. In another example, the display device 107a may cache some or all of the analyte data and the analyte sensor system 104 may cache less data than the display device 107a and/or may cache less data than the analyte sensor system 104 normally caches (e.g., less metadata than is normally cached). In addition, or alternatively, caching responsibility may be split between the display device 107a and the analyte sensor system 104, for example, such that the display device 107a caches a first portion of the analyte data and the analyte sensor system 104 caches a second portion of the analyte data that is different from the first portion.

At decision block 606, the application 106 determines whether the display device 107 is currently connected to the other device. If it is determined, at the decision block 606, that the display device 107 is not currently connected to the other device, the process 600 returns to the block 602 and executes as described previously. However, if it is determined, at the decision block 606, that the display device 107 is currently connected to the other device, the process 600 proceeds to block 608.

At block 608, the application 106 causes the cached analyte data to be transmitted to the other device. In some cases, the determination that the display device 107 is currently connected to the other device can be a condition sufficient for termination of the process 600, or for switching to a different device-to-device transmission flow (e.g., the process 500 of FIG. 5A), for example, during a parallel execution of the process 400 of FIG. 4A, as discussed above. However, if the process 600 is not terminated, from block 608, the process 600 returns to the block 602 and executes as described previously. In various aspects, the process 600 can continue until conditions for device-to-device transmission are no longer satisfied, for example, according to the process 400 of FIG. 4A. In addition, or alternatively, the process 600 can continue until a command to terminate the process 600 is received, for example, according to the process 450 of FIG. 4B.

In certain aspects, caching functionality as described relative to the process 600 of FIG. 6A can be configured to optimize, for example, resources of the analyte sensor system 104. For example, in some aspects, the display device 107 may cache more data than the analyte sensor system 104 as a design decision to save storage on the analyte sensor system 104. In some aspects, when the display device 107 is caching data, the analyte sensor system 104 may not cache such data, or may cache only a portion of the data (e.g., cache only a summary of the data, cache less metadata than it normally caches, etc.).

In certain aspects, the analyte sensor system 104 and the display device 107 can combine or coordinate to provide analyte data to the other device experiencing the communication disruption. In some cases, the same underlying issue may cause the other device to lose its connectivity to both the analyte sensor system 104 and the display device 107 (e.g., the other device may have powered off due to a depleted battery or may have been left behind during an activity, such as running or swimming). In such cases, the other device may reestablish connectivity with the analyte sensor system 104 and the display device 107 at the same time or at approximately the same time. Accordingly, in certain aspects, if the other device reestablishes connections with both the display device 107 and the analyte sensor system 104, the display device 107 and/or the analyte sensor system 104 can coordinate to provide cached analyte data to the other device.

Continuing the above example, in certain aspects, a transmission hierarchy can be implemented to determine whether cached analyte data is transmitted to the other device by the analyte sensor system 104, the display device 107, or both the analyte sensor system 104 and the display device 107. In an example, the transmission hierarchy can be implemented on the other device, such that that the other device requests missing analyte data from a device indicated by the hierarchy. In another example, the transmission hierarchy can specify that the display device 107 is responsible for transmitting cached analyte data to the other device, which has the advantage of saving battery, memory and processing at the analyte sensor system 104. In yet another example, the transmission hierarchy can consider dynamic attributes of one or both of the display device 107 and the analyte sensor system 104. For example, if the display device 107 has a battery level below a predetermined threshold, the analyte sensor system 104 can transmit cached analyte data to the other device. Likewise, if the analyte sensor system 104 has a battery level below a predetermined threshold, the display device 107 can transmit cached analyte data to the other device.

FIG. 6B is a sequence diagram 650 illustrating example interactions between the analyte sensor system 104, the display device 107c, and the display device 107a, in accordance with certain embodiments. The sequence diagram 650 will be described in relation to the process 400 of FIG. 4A, the process 450 of FIG. 4B, and the process 600 of FIG. 6A.

Steps 652 and 654 correspond to a period before initiation of a device-to-device transmission flow, such as the process 600 of FIG. 6A. At steps 652 and 654, the analyte sensor system 104 transmits analyte data directly to the display device 107c and the display device 107a, respectively. Concurrent with the steps 652 and 654, the display devices 107a and 107c may each continuously execute a process for device-to-device mitigation of disrupted communication with analyte sensors, such as the process 400 of FIG. 4A discussed above. In addition, or alternatively, the display devices 107a and 107c may each execute a process for device-to-device mitigation based on received indications of communication disruptions (e.g., commands), such as the process 450 of FIG. 4B discussed above.

At step 656, the display device 107c loses connectivity with the analyte sensor system 104, thus beginning a period of communication disruption with the analyte sensor system 104. Additionally, at step 658, which may be concurrent with the step 658, the display device 107c loses connectivity with the display device 107a, thus beginning a period of communication disruption with the display device 107a. Subsequent to the steps 656 and 658, with reference to the process 400 of FIG. 4A, the display device 107a can determine a current communication status of the display device 107c. Alternatively, subsequent to the steps 656 and 658, with reference to the process 450 of FIG. 4B, the display device 107a can receive an indication of a change in communication status of the display device 107c. According to the example of FIG. 6B, the current communication status, or the received indication of a change in communication status, may indicate that the display device 107c is: a) not currently connected to the analyte sensor system 104; and b) not currently connected to the display device 107a. As discussed previously, the received indication of a change in communication status may be, for example, a command to initiate a device-to-device transmission flow, such as the process 600 of FIG. 6A. In response to the current communication status of the display device 107c, or the received indication of the change in communication status, the display device 107a can initiate the process 600 of FIG. 6A so as to cache analyte data for later transmission to the display device 107c.

Steps 660, 662, and 664 correspond to a period during execution of the process 600 of FIG. 6A. At step 660, the analyte sensor system 104 transmits analyte data directly to the display device 107a, in similar fashion to the step 654 discussed above, in response to which the display device 107a caches the received analyte data, as discussed above relative to the process 600. Step 660, and the corresponding caching, can repeat until a connection with the display device 107c is reestablished or until a termination command is received, as discussed previously.

At steps 662 and 663, the display device 107c reestablishes connections with the display device 107a and the analyte sensor system 104, respectively. Subsequent to the steps 662 and 663, with reference to the process 400 of FIG. 4A, the display device 107a can determine a current communication status of the display device 107c. Alternatively, subsequent to the steps 662 and 663, with reference to the process 450 of FIG. 4B, the display device 107a can receive an indication of a change in communication status of the display device 107c. According to the example of FIG. 6B, the current communication status, or the received indication of a change in communication status, may indicate that the display device 107c is once again connected to both the analyte sensor system 104 and the display device 107a. As discussed previously, the received indication of a change in communication status may be, for example, a command to terminate the device-to-device transmission flow, such as the process 600 of FIG. 6A. At step 664, the display device 107a transmits the cached analyte data to the display device 107a. In addition, in response to the current communication status of the display device 107c, or the received indication of the change in communication status, the display device 107a can terminate the process 600 of FIG. 6A.

Steps 666 and 668 correspond to a period after termination of a device-to-device transmission flow, such as the process 600 of FIG. 6A. At steps 666 and 668, the analyte sensor system 104 transmits analyte data directly to the display device 107c and the display device 107a, respectively, as discussed relative to the steps 652 and 654.

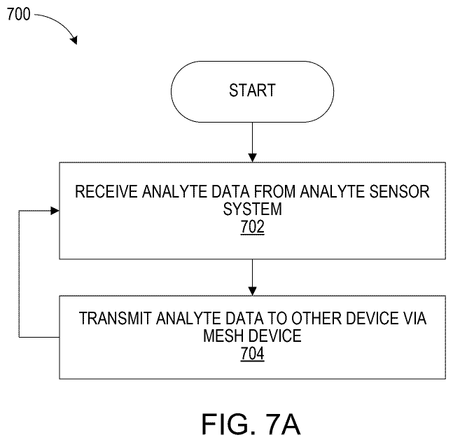

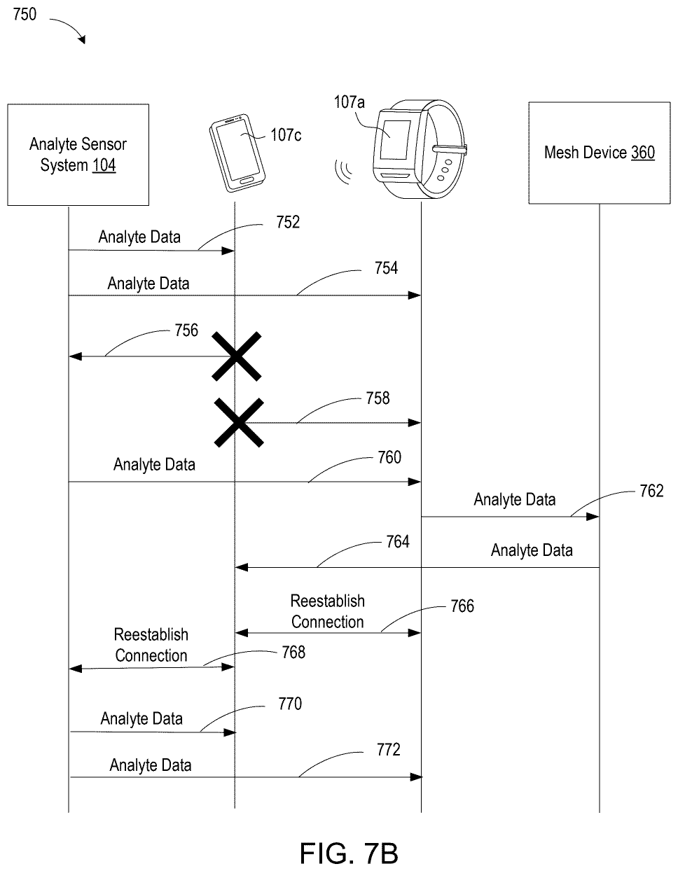

FIG. 7A illustrates an example of a process 700 for executing a device-to-device transmission flow via a mesh network, in accordance with certain embodiments. In some embodiments, the process 700 can be executed, for example, by the therapy management engine 112 of FIGS. 1A-B and 2. In addition, or alternatively, the process 700 can be executed, for example, by the application 106 of FIGS. 1A-B and 2. In addition, or alternatively, the process 700 can be executed generally by any of the display devices 107 of FIGS. 1A-B and 2. Although any number of systems, in whole or in part, can implement the process 700, to simplify discussion, the process 700 will be described as being performed by the application 106 of FIGS. 1A-B and 2, with particular reference to the system 300 of FIG. 3.

In certain aspects, the process 700 can be initiated as part of the process 400 of FIG. 4A, for example, if a current communication status of another device indicates that the other device is not currently connected to the analyte sensor system 104 and also that the other device is currently connected to the display device 107. In addition, or alternatively, the process 700 can be initiated as part of the process 450 of FIG. 4B, for example, in response to a received indication of a change in communication status of the other device (e.g., a command). In some aspects, the process 700 can execute at the display device 107 in parallel to repeated, or continuous, execution of the process 400 of FIG. 4A.

At block 702, the application 106 receives analyte data from the analyte sensor system 104, in similar fashion to the block 402 of FIG. 4A. In general, the block 702 is illustrative of analyte data to be processed for device-to-device transmission according to the process 700 of FIG. 7A. In certain aspects, the block 702 is a reference to the receipt of analyte data according to the block 402 of FIG. 4A or the block 452 of FIG. 4B. In certain aspects, the application 106 can cache or buffer the analyte data on the display device 107, for example, until block 704 is executed.

At block 704, the application 106 causes the display device 107 to transmit the analyte data to the other device via the mesh device 360, for example, in real-time as the analyte data is received from the analyte sensor system 104. As discussed previously, the mesh device 360 can be representative of a plurality of mesh devices utilized to relay information over a mesh network. In some aspects, as discussed previously, the display device 107 can be the mesh device 360, or one of the mesh devices 360. In some aspects, the mesh device 360 can be selected, for example, based on an analysis of a connection list, as generally discussed relative to FIGS. 3 and 4A. From block 704, the process 700 returns to the block 702 and executes as described previously. In various aspects, the process 700 can continue until conditions for device-to-device transmission are no longer satisfied, for example, according to the process 400 of FIG. 4A. In addition, or alternatively, the process 700 can continue until a command to terminate the process 700 is received, for example, according to the process 450 of FIG. 4B.

FIG. 7B is a sequence diagram 750 illustrating example interactions between the analyte sensor system 104, the display device 107c, the display device 107a, and the mesh device 360, in accordance with certain embodiments. In particular, the sequence diagram 750 illustrates an example scenario in which the mesh device 360 may be used to provide an entry point to a mesh network, for example, in response to a communication disruption. In various aspects, the mesh device 360 can thereby provide a protocol stack for relaying, for example, analyte data to a device experiencing the communication disruption.

Steps 752 and 754 correspond to a period before initiation of a device-to-device transmission flow. At steps 752 and 754, the analyte sensor system 104 transmits analyte data directly to the display device 107c and the display device 107a, respectively. Concurrent with the steps 752 and 754, the display devices 107a and 107c may each continuously execute a process for device-to-device mitigation of disrupted communication with analyte sensors, such as the process 400 of FIG. 4A discussed above. In addition, or alternatively, the display devices 107a and 107c may each execute a process for device-to-device mitigation based on received indications of communication disruptions (e.g., commands), such as the process 450 of FIG. 4B discussed above.

At step 756, the display device 107c loses connectivity with the analyte sensor system 104, thus beginning a period of communication disruption with the analyte sensor system 104. Additionally, at step 758, which may be concurrent with the step 658, the display device 107c loses connectivity with the display device 107a, thus beginning a period of communication disruption with the display device 107a. Subsequent to the steps 756 and 758, with reference to the process 400 of FIG. 4A, the display device 107a can determine a current communication status of the display device 107c. Alternatively, subsequent to the steps 756 and 758, with reference to the process 450 of FIG. 4B, the display device 107a can receive an indication of a change in communication status of the display device 107c. According to the example of FIG. 7B, the current communication status, or the received indication of a change in communication status, may indicate that the display device 107c is: a) not currently connected to the analyte sensor system 104; and b) not currently connected to the display device 107a. In response to the current communication status of the display device 107c, or the received indication of the change in communication status, if the display device 107a determines that a mesh network is available, and that the display device 107c is reachable over the mesh network, the display device 107a can initiate a device-to-device transmission flow utilizing the mesh device 360. In various aspects, the device-to-device transmission flow may correspond to the process 700 of FIG. 7A.

Steps 760-768 correspond to a period during execution of the device-to-device transmission flow. At step 760, the analyte sensor system 104 transmits analyte data directly to the display device 107a, in similar fashion to the step 754 discussed above. At step 762, the display device 107a transmits the analyte data to the mesh device 360. At step 764, the mesh device 360 transmits or relays the analyte data to the display device 107c via the mesh network. Steps 760-764 can repeat until connections between the display device 107c and one or both of the analyte sensor system 104 and the display device 107c are reestablished, or until a termination command is received, as discussed previously.

At steps 766 and 768, the display device 107c re-establishes connections with the display device 107a and the analyte sensor system 104, respectively. Subsequent to the steps 766 and 768, with reference to the process 400 of FIG. 4A, the display device 107a can determine a current communication status of the display device 107c. Alternatively, subsequent to the steps 766 and 768, with reference to the process 450 of FIG. 4B, the display device 107a can receive an indication of a change in communication status of the display device 107c. According to the example of FIG. 6B, the current communication status, or the received indication of a change in communication status, may indicate that the display device 107c is once again connected to both the analyte sensor system 104 and the display device 107a. As discussed previously, the received indication of a change in communication status may be, for example, a command to terminate the device-to-device transmission flow. In response to the current communication status of the display device 107c, or the received indication of the change in communication status, the display device 107a can terminate the device-to-device transmission flow.

Steps 770 and 772 correspond to a period after termination of the device-to-device transmission flow. At steps 770 and 772, the analyte sensor system 104 transmits analyte data directly to the display device 107c and the display device 107a, respectively, as discussed relative to the steps 752 and 754.

For illustrative purposes, FIGS. 7A-B describes example utilization of a mesh network. It should be appreciated, however, that a communication path to the display device 107c, for example, can further include one or more cloud platforms, one or more communication or messaging services, and/or the like.

FIG. 8 illustrates an example of a process 800 for anticipatory mitigation of a predicted communication disruption, in accordance with certain embodiments. In some embodiments, the process 800 can be executed, for example, by the therapy management engine 112 of FIGS. 1A-B and 2. In addition, or alternatively, the process 800 can be executed, for example, by the application 106 of FIGS. 1A-B and 2. In addition, or alternatively, the process 800 can be executed generally by any of the display devices 107 of FIGS. 1A-B and 2.

Although any number of systems, in whole or in part, can implement the process 800, to simplify discussion, the process 800 will be described as being performed by the application 106 of FIGS. 1A-B and 2, with particular reference to the system 300 of FIG. 3. With reference to the system 300 of FIG. 3, in some aspects, the process 800 can be executed continuously by each of the applications 106-1 and 106-2. In addition, or alternatively, the process 800 can be executed by the application 106-1 and the application 106-2 at any suitable interval (e.g. every 5 minutes, every 10 minutes, every 15 minutes, etc.). Other variations will be apparent to one skilled in the art after a detailed review of the present disclosure.

At block 802, at the display device 107, the application 106 receives analyte data (e.g., analyte measurements) from the analyte sensor system 104. The analyte data may be received, for example, via the transceiver 136 of the analyte sensor system 104, as discussed relative to FIGS. 1A-B and 3.

At block 804, the application 106 processes the analyte data at the display device 107. In general, the block 804 can include the application 106 executing logic to store, analyze and/or display the analyte data according to its standard application flow. In addition, or alternatively, the block 804 can include, for example, generating and/or presenting alerts based on the analyte data (e.g., high, low, rapidly increasing, rapidly decreasing, etc.).

At block 806, the application 106 determines a predicted communication status of another device. In general, the predicted communication status characterizes a future ability of the other device to communicate with the analyte sensor system 104 and/or the display device 107. In an example, if the process 800 is executed by the application 106-1 executing on the display device 107a, the block 806 can include determining a predicted communication status of the display device 107c. In another example, if the process 800 is executed by the application 106-2 executing on the display device 107c, the block 806 can include determining a predicted communication status of the display device 107a.

In certain aspects, the predicted communication status can characterize a connection between the other device and the analyte sensor system 104. The predicted communication status can include, for example, an indication of whether the other device is predicted to lose connectivity with the analyte sensor system 104 within a specified period of time (e.g., within the next 15 minutes, one hour, etc.). In certain aspects, the predicted communication status can include, for example, predicted dynamic attributes of the other device and/or the connection between the other device and the analyte sensor system 104. The predicted dynamic attributes can include, for example, a signal strength associated with the other device's connection with the analyte sensor system 104, a power level (e.g., battery level) of the other device, combinations of the foregoing and/or the like.

In certain aspects, the predicted communication status can be based on time of day and/or historical location data, such as global positioning system (GPS) data used to track a location of the display device 107 and/or a location of the other device, where the historical location data is timestamped (e.g., mapped to time of day, day of week, date, etc.). For example, the other device (e.g., smartphone) may experience a communication disruption every Tuesday at noon at the North Park Aquatic Center (e.g., while the user 102 is swimming). The location of the North Park Aquatic Center may be indicated, for example, by location data associated with the display device 107 and/or the other device. According to this example, at a configurable predetermined time prior to each Tuesday at noon (e.g., 15 minutes beforehand), the predicted communication status may indicate a predicted loss of connectivity between the analyte sensor system 104 and the other device. In addition, or alternatively, in response to current location data indicating that the display device 107 and/or the other device is currently approaching, or at, the North Park Aquatic Center, the predicted communication status may likewise indicate a predicted loss of connectivity between the analyte sensor system 104 and the other device, regardless of day and time.

In addition, or alternatively, the predicted communication status can be based on current dynamic attributes of the other device and/or of the connection between the other device and the analyte sensor system. The current dynamic attributes can include, for example, a signal strength associated with the connection or a power level (e.g., battery level) of the smartphone. In general, the current dynamic attributes can be determined and used in any of the ways described relative to the block 406 of FIG. 4A. In some cases, the current dynamic attributes may be sufficient to determine a predicted communication disruption, although they may not be sufficient to constitute a current communication disruption, for example, according to the process 400 of FIG. 4A.

In addition, or alternatively, the predicted communication status can be based on similarity of data or conditions to data or conditions associated with previous periods of disruption. For example, previous periods of disruption can be identified and linked to time, day, location, location type, and/or dynamic attributes of the type discussed above. For example, if a communication disruption occurs every time (or nearly every time) the display device 107 and/or the other device approaches a beach location, the predicted communication status may indicate a predicted loss of connectivity between the analyte sensor system 104 and the other device, even if the beach location is not included, for example, in historical location data.

At decision block 808, the application 106 determines, based on the predicted communication status of the other device, whether one or more conditions for anticipatory mitigation are satisfied. In an example, the condition(s) can be satisfied if the predicted communication status indicates that the other device is predicted to lose connectivity with the analyte sensor system 104 within a specified period of time (e.g., 15 minutes, 30 minutes, etc.), for example, based on any of the data discussed previously.

If it is determined, at the decision block 808, that the conditions(s) for anticipatory mitigation are satisfied, at block 810, the application 106 initiates anticipatory mitigation measures, such as anticipatory caching of analyte data. As mentioned above, in some aspects, the initiating can occur at a configurable predetermined time prior to a time at which a communication disruption is predicted to occur, such as when the predicted communication status is based on time of day (e.g., caching can begin 15 minutes prior to a daily noon visit to a pool). In some aspects, anticipatory can occur immediately, such as when the predicted communication status is based on current dynamic attributes (e.g., location data indicating an approach to a pool or beach, as discussed above). In some cases, the initiating can involve updating a corresponding setting, for example, by turning the setting to “ON.” In some aspects, if anticipatory caching is already initiated, for example, due to a command or a previous iteration of the process 800, the anticipatory mitigation is allowed to continue execution.

In an example, the anticipatory mitigation can involve the display device 107 taking action to proactively cache analyte data, even if the other device is currently connected to the analyte sensor system 104. For example, the application 106 on the display device 107 can initiate caching, for example, of the type discussed relative to FIGS. 5A and 5B. In this way, if the communication disruption later occurs, or if the communication disruption has already occurred but has not yet been discovered by the display device 107, the anticipatory caching can reduce or eliminate delays in the initiation of caching. In various aspects, the cached analyte data on the display device 107 is a more complete dataset than would be present if caching were only initiated reactively in response to an actual communication disruption. Therefore, in various aspects, by virtue of enacting anticipatory caching, the display device 107 is better able to handle device-to-device transmission of data to the other device (e.g., according to device-to-device transmission flows, such as the process 600 of FIG. 6A, or upon connection reestablishment), thereby further relieving the analyte sensor system 104 of caching and/or transmission duties in the ways discussed previously.

In some aspects, the anticipatory mitigation can involve temporarily changing operation of the analyte sensor system 104, such as by causing the transceiver 136 to temporarily boost power, for example, to accommodate a less sensitive transceiver of the display device 107 as compared to the other device, if applicable (e.g., +4 dB, if the display device 107 is smartwatch rather than a smartphone). In certain aspects, the anticipatory mitigation measures can continue until the condition(s) for anticipatory mitigation are no longer satisfied.

If it is determined, at the decision block 808, that the conditions(s) for anticipatory mitigation are not satisfied, at block 812, the application 106 terminates the anticipatory mitigation measures, if any such measures are active or executing. If no anticipatory mitigation measures are active or executing, the non-execution of such measures is allowed to continue. In certain aspects, termination of the anticipatory mitigation measures can involve updating a setting, for example, by turning the setting to “OFF.” In addition, or alternatively, if operation of the analyte sensor system 104 was temporarily changed at the block 810 (e.g., by boosting power of the transceiver 136), the termination can involve restoring previous or standard operation of the analyte sensor system 104. After either block 810 or 812, the process 800 ends.

For illustrative purposes, the process 800 is described as initiating or terminating anticipatory mitigation based on a predicted communication status of another device. With reference to FIG. 3, in certain aspects, anticipatory mitigation can also be initiated or terminated in response to a command that is received, for example, from the other device (e.g., the display device 107a or the display device 107c), the mesh device 360, the supporter device 311, the server system 330, and/or the like. In some aspects, the command be a user command, for example, from the user 102 or the supporter 308. In certain aspects, the command can specify or be associated with a termination condition, such as a period of time (e.g., 30 minutes, 2 hours, etc.), a device location (e.g., while the display device 107a is at home, away from home, at a pool, etc.). Accordingly, the command can remain in effect until the termination condition is satisfied. Other examples of triggers for initiating and/or terminating anticipatory mitigation will be apparent to one skilled in the art after a detailed review of the present disclosure.

In some aspects, anticipatory mitigation as discussed above can be implemented in a way that maximizes battery life of the display device 107. For example, if the display device 107 has a battery level below a predetermined threshold, the display device 107 may not enact anticipatory mitigation, thereby leaving caching duties with the analyte sensor system 104.

In certain aspects, a device that experiences a communication disruption, such as the other device of FIGS. 5A and 6A and/or the display device 107c in the example of FIGS. 5B, 6B and 7, may receive overlapping analyte data from multiple sources upon connection reestablishment. In certain aspects, analyte data that is shared or communicated between devices can involve tracking a state of each device and maintaining health data identifiers, such as timestamps, transmitter/source identifier, etc. to prevent duplicate data. In certain aspects, the foregoing data can be utilized, for example, to de-duplicate analyte data.

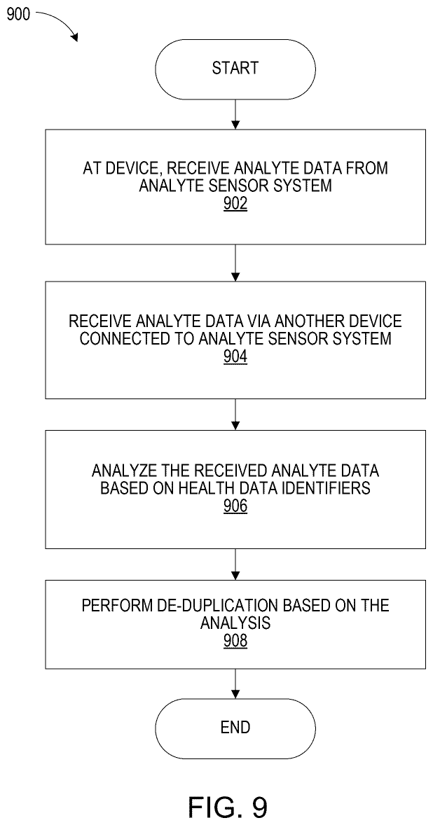

FIG. 9 illustrates an example of a process 900 for de-duplicating data, in accordance with certain embodiments. In certain aspects, the process 900 can be executed, for example, by a device that receives analyte data during or after a communication disruption, for example, with the analyte sensor system 104. In some embodiments, the process 900 can be executed, for example, by the therapy management engine 112 of FIGS. 1A-B and 2. In addition, or alternatively, the process 900 can be executed, for example, by the application 106 of FIGS. 1A-B and 2. In addition, or alternatively, the process 900 can be executed generally by any of the display devices 107 of FIGS. 1A-B and 2. Although any number of systems, in whole or in part, can implement the process 900, for simplicity of description, the process 900 will be described as being performed by the display device 107c of FIG. 3.

At block 902, the display device 107c receives analyte data from the analyte sensor system 104. At block 904, the display device 107c receives analyte data via another device connected to the analyte sensor system 104, such as the display device 107a and/or the mesh device 360. In various aspects, the analyte data received at the blocks 902 and 904 can follow, and/or occur during, a communication disruption with the analyte sensor system 104. The analyte data received at the blocks 902 and 904 can include, for example, health data identifiers as discussed above.

At block 906, the display device 107c analyzes the analyte data received at the blocks 902 and 904 based on the health data identifiers. The block 906 can include, for example, identifying matching health data identifiers between the analyte data received at the at the block 902 and the analyte data received at the block 904. At block 908, the display device performs de-duplication based on the analysis at block 906. For example, the display device 107c can remove or delete redundant analyte data based on the analysis at the block 906, and can fill in missing analyte data. After block 908, the process 900 ends.

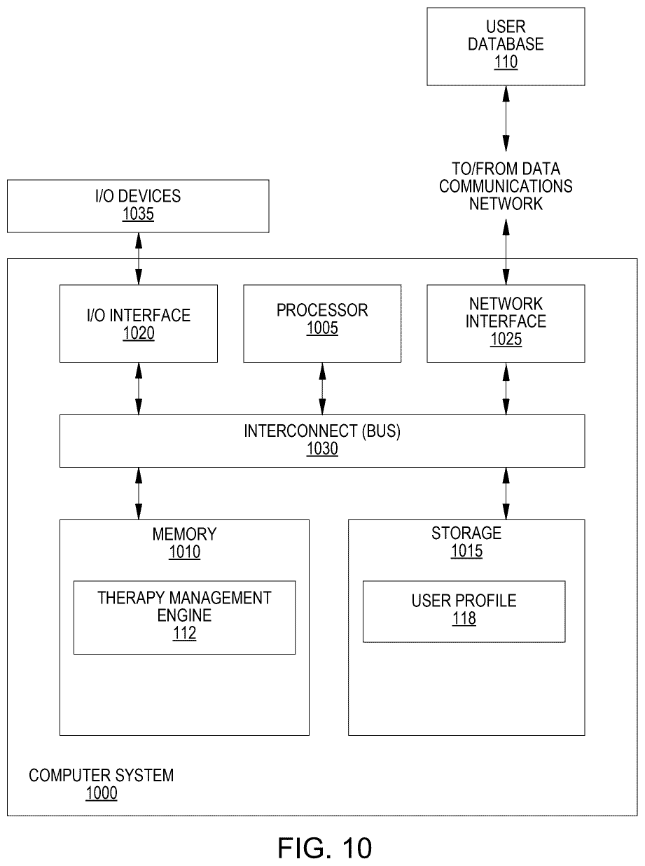

FIG. 10 is a block diagram depicting a computer system 1000 configured for optimizing transmission of analyte data in multi-receiver environments, for example, according to certain embodiments disclosed herein. Although depicted as a single physical device, in embodiments, the computer system 1000 may be implemented using virtual device(s), and/or across a number of devices, such as in a cloud environment and/or via separate modules of portable or cloud devices. As illustrated, the computer system 1000 includes a processor 1005, a memory 1010, a storage 1015, a network interface 1025, and one or more I/O interfaces 1020. In the illustrated embodiment, the processor 1005 retrieves and executes programming instructions stored in the memory 1010, as well as stores and retrieves application data residing in the storage 1015. The processor 1005 is generally representative of a single CPU and/or GPU, multiple CPUs and/or GPUs, a single CPU and/or GPU having multiple processing cores, and the like.

The memory 1010 is generally included to be representative of a random access memory (RAM). The storage 1015 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, the I/O devices 1035 (such as keyboards, monitors, etc.) can be connected via the I/O interface(s) 1020. Further, via the network interface 1025, the computer system 1000 can be communicatively coupled with one or more other devices and components, such as the user database 110. In certain embodiments, the computer system 1000 is communicatively coupled with other devices via a network, which may include the Internet, local network(s), and the like. The network may include wired connections, wireless connections, or a combination of wired and wireless connections. As illustrated, the processor 1005, memory 1010, storage 1015, network interface(s) 1025, and the I/O interface(s) 1020 are communicatively coupled by one or more interconnects 1030. In certain embodiments, the computer system 1000 is representative of the display device 107 associated with the user. In certain embodiments, as discussed above, the display device 107 can include the user's laptop, computer, smartphone, and the like. In another embodiment, the computer system 1000 is a server executing in a cloud environment.

In the illustrated embodiment, the storage 1015 includes the user profile 118. The memory 1010 includes the therapy management engine 112. The therapy management engine 112 can be executed by the computer system 1000 to perform operations, for example, of the process 400 of FIG. 4A, the process 450 of FIG. 4B, the process 500 of FIG. 5A, the sequence diagram 550 of FIG. 5B, the process 600 of FIG. 6A, the sequence diagram 650 of FIG. 6B, the process 700 of FIG. 7A, the sequence diagram 750 of FIG. 7B, the process 800 of FIG. 8, and/or the process 900 of FIG. 9.

In certain aspects, a method of mitigating disrupted communication with an analyte sensor includes receiving, at a first device, analyte measurements from the analyte sensor. The method also includes receiving, at the first device, an indication of a communication disruption between the analyte sensor and a second device. The method also includes, responsive to the indication, at the first device, facilitating transfer of at least a portion of the analyte measurements from the first device to the second device. Certain embodiments of these aspects include corresponding computer systems, apparatus, and computer programs recorded on one or more computer storage devices, each configured to perform the actions of the methods.

In certain aspects, a method of mitigating disrupted communication with an analyte sensor includes receiving, at a first device, analyte measurements from the analyte sensor. The method also includes determining a communication status of a second device. The method also includes, responsive to the communication status indicating a communication disruption between the second device and the analyte sensor, at the first device, facilitating transfer of at least a portion of the analyte measurements from the first device to the second device. Certain embodiments of these aspects include corresponding computer systems, apparatus, and computer programs recorded on one or more computer storage devices, each configured to perform the actions of the methods.

In certain aspects, a method of mitigating disrupted communication with an analyte sensor includes receiving, at a first device, analyte measurements from the analyte sensor. The method also includes receiving, at the first device, an indication of a communication disruption between the analyte sensor and a second device. The method also includes transmitting at least a portion of the analyte measurements to the second device as the analyte measurements are received from the analyte sensor. Certain embodiments of these aspects include corresponding computer systems, apparatus, and computer programs recorded on one or more computer storage devices, each configured to perform the actions of the methods.

In certain aspects, a method of mitigating disrupted communication with an analyte sensor includes receiving, at a first device, analyte measurements from the analyte sensor. The method also includes receiving, at the first device, an indication of a communication disruption between the analyte sensor and a second device. The method also includes transmitting at least a portion of the analyte measurements to the second device over a first connection as the analyte measurements are received from the analyte sensor. The method also includes identifying, at the first device, a performance consideration related to communication between the first device and the second device over the first connection. The method also includes identifying, at the first device, a second connection for communication with the first device based on the performance consideration. The method also includes transmitting at least a portion of the analyte measurements to the second device over the second connection as the analyte measurements are received from the analyte sensor. Certain embodiments of these aspects include corresponding computer systems, apparatus, and computer programs recorded on one or more computer storage devices, each configured to perform the actions of the methods.

In certain aspects, a method of mitigating disrupted communication with an analyte sensor includes receiving, at a first device, analyte measurements from the analyte sensor. The method also includes receiving, at the first device, an indication of a communication disruption between a second device and each of the analyte sensor and the first device. The method also includes, responsive to the indication, at the first device, caching at least a portion of the analyte measurements at the first device for later transmission to the second device. The method also includes, responsive to a determination that the second device is connected to the first device, transmitting the cached at least a portion of the analyte measurements to the second device. Certain embodiments of these aspects include corresponding computer systems, apparatus, and computer programs recorded on one or more computer storage devices, each configured to perform the actions of the methods.

In certain aspects, a method of mitigating disrupted communication with an analyte sensor includes receiving, at a first device, analyte measurements from the analyte sensor. The method also includes receiving, at the first device, an indication of a communication disruption between the analyte sensor and a second device. The method also includes transmitting at least a portion of the analyte measurements to the second device via a mesh network as the analyte measurements are received from the analyte sensor. Certain embodiments of these aspects include corresponding computer systems, apparatus, and computer programs recorded on one or more computer storage devices, each configured to perform the actions of the methods.

In certain aspects, a method of mitigating disrupted communication with an analyte sensor includes receiving, at a first device, analyte measurements from the analyte sensor. The method also includes determining a predicted communication status of a second device, the predicted communication status indicating that a communication disruption is predicted to occur within a specified period of time. The method also includes, responsive to the predicted communication status indicating a predicted communication disruption between the second device and the analyte sensor, at the first device, initiating caching of at least a portion of the analyte measurements. Certain embodiments of these aspects include corresponding computer systems, apparatus, and computer programs recorded on one or more computer storage devices, each configured to perform the actions of the methods.

Each of these non-limiting examples can stand on its own or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method of mitigating disrupted communication with an analyte sensor, the method comprising:
receiving, at a first device, analyte measurements from the analyte sensor;
receiving, at the first device, an indication of a communication disruption between the analyte sensor and a second device; and
responsive to the indication, at the first device, facilitating transfer of at least a portion of the analyte measurements from the first device to the second device.

2. The method of claim 1, wherein the facilitating transfer comprises transmitting the at least a portion of the analyte measurements to the second device as the analyte measurements are received from the analyte sensor.

3. The method of claim 2, wherein the transmitting comprises direct communication between the first device and the second device.

4. The method of claim 2, further comprising:
determining, at the first device, a loss of connectivity between the first device and the second device over a first connection; and
responsive to the determining, identifying, at the first device, a second connection for communication with the first device, wherein the transmitting comprises utilizing the second connection.

5. The method of claim 2, wherein the transmitting comprises utilizing a mesh network.

6. The method of claim 1, wherein the indication further indicates a communication disruption between the first device and the second device, and the facilitating transfer comprises caching the at least a portion of the analyte measurements at the first device for later transmission to the second device.

7. The method of claim 6, wherein the caching comprises caching the at least a portion of the analyte measurements at a greater level of granularity than the analyte sensor.

8. The method of claim 6, further comprising, responsive to a determination that the second device is connected to the first device, transmitting the cached at least a portion of the analyte measurements to the second device.

9. The method of claim 6, further comprising, responsive to a determination that the second device is connected to the first device, coordinating with the analyte sensor to transmit the cached at least a portion of the analyte measurements to the second device.

10. The method of claim 9, wherein the coordinating comprises, responsive to a determination that the first device is higher than the analyte sensor in a transmission hierarchy, transmitting the cached at least a portion of the analyte measurements to the second device.

11. The method of claim 1, wherein the indication comprises a command to initiate the facilitated transfer of at least a portion of the analyte measurements from the first device to the second device.

12. The method of claim 1, further comprising presenting information related to the analyte measurements at the first device.

13. A method of mitigating disrupted communication with an analyte sensor, the method comprising:
receiving, at a first device, analyte measurements from the analyte sensor;
determining a communication status of a second device; and
responsive to the communication status indicating a communication disruption between the second device and the analyte sensor, at the first device, facilitating transfer of at least a portion of the analyte measurements from the first device to the second device.

14. The method of claim 13, wherein the communication disruption comprises a technical problem at the second device.

15. The method of claim 13, wherein the communication status is a current communication status of the second device.

16. The method of claim 13, wherein:
the communication status is a predicted communication status indicating that the communication disruption is predicted to occur within a specified period of time; and
the facilitating transfer comprises initiating caching of the at least a portion of the analyte measurements.

17. The method of claim 16, wherein the predicted communication status is determined based on at least one of current time or current location.

18. The method of claim 13, further comprising, responsive to the communication status indicating the communication disruption, boosting power of a transmitter associated with the analyte sensor.

19. A system for mitigating disrupted communications, comprising:
an analyte sensor configured to generate measurements associated with an analyte level of a patient;
a device in communication with the analyte sensor and configured to:
receive analyte measurements from the analyte sensor;
receive an indication of a communication disruption between the analyte sensor and another device; and
responsive to the indication, facilitate transfer of at least a portion of the analyte measurements from the device to the other device.

20. A computer-program product comprising a non-transitory computer-usable medium having computer-readable program code embodied therein, the computer-readable program code adapted to be executed to implement a method of mitigating disrupted communication with an analyte sensor, the method comprising:
receiving, at a first device, analyte measurements from the analyte sensor;
receiving, at the first device, an indication of a communication disruption between the analyte sensor and a second device; and
responsive to the indication, at the first device, facilitating transfer of at least a portion of the analyte measurements from the first device to the second device.
